(19) United States
(12) Patent Application Publication
(10) Pub. No.: US 2005/0187240 A1
(43) Pub. Date: Aug. 25, 2005

(54) Aaptamine and isoaaptamine and structural modifications thereof

(75) Inventors: George R. Pettit, Paradise Valley, AZ (US); Delbert L. Herald, Tempe, AZ (US); Holger Hoffman, Heppenheim (DE)

Correspondence Address: FENNEMORE CRAIG, 3003 NORTH CENTRAL AVENUE, SUITE 2600, PHOENIX, AZ, 85012, US

(73) Assignee: The Arizona Board of Regents, Acting for and on behalf of the Arizona State University

(21) Appl. No.: 11/064,654

(22) Filed: Feb. 23, 2005

Related U.S. Application Data
(60) Provisional application No. 60/547,245, filed on Feb. 23, 2004.

(51) Int. Cl. A61K 31/4745; C07D 471/02

(52) U.S. Cl. 514/283; 514/291; 546/48; 546/81

(57) ABSTRACT

Disclosed herein is the isolation and elucidation of the structure of isoaptamine (2), and its conversion to numerous novel related compounds, which appear to have antimicrobial and/or cancer fighting properties. Also disclosed is the conversion of isoaaptamine (2) to the phosphate prodrug hystatin 1 (11), as well as the conversion of aaptamine to numerous compounds, including but not limited to: 9-demethyloxyaaptamine (3); 4-methylaaptamine (4); 1,4-dimethylaaptamine iodide (5); 4-N-methyl-8,9-dihydroxy-4H benzo[de][1,6]naphthyridine (7); 4-N-methyl-8 methoxy-9 hydroxy-4H benzo[de][1,6]naphthyridine (8); 1-H-Benzo[de][1-6]naphthyridinium salts (9a-c); hystatin 2 (10a); and others.

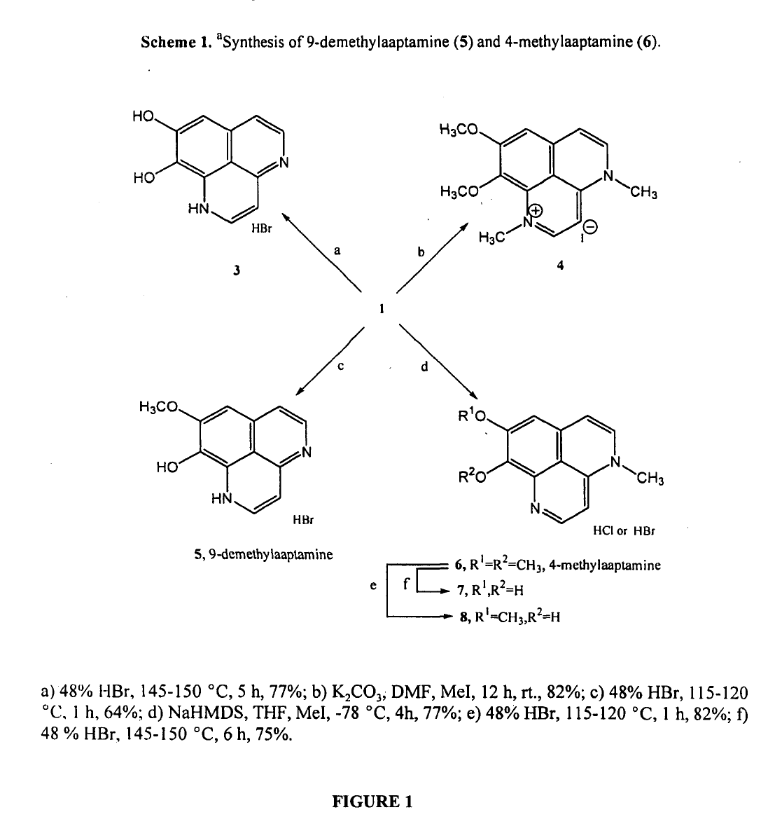

### RELATED APPLICATION DATA

This application is based on and claims the benefit of U.S. Provisional Patent Application No. 60/547,245 filed on Feb. 23, 2004, the disclosure of which is incorporated herein in its entirety by this reference.

### INTRODUCTION

Financial assistance for this invention was provided by the United States Government, Division of Cancer Treatment and Diagnosis, National Cancer Institute, Department of Health and Human Services, Outstanding Investigator Grant Numbers CA44344-01-12, CA44344-01A1-12, CA 44344-05-12 and CA90441-01; the Arizona Disease Control Research Commission; and private contributions. Thus, the United States Government has certain rights in this invention.

### FIELD OF THE INVENTION

This invention relates to novel compounds, and methods for isolation and/or synthesis thereof, many of which exhibit significant anticancer and antimicrobial activities.

### BACKGROUND OF THE INVENTION

The isolation and structural determination of new anticancer constituents from marine invertebrates continues to increase. Marine porifera, or sponges, continue to be a source of new drug candidates having very important biological activities. In 1992, during a marine invertebrate survey in the Republic of Singapore, the inventors located a previously unknown species of sponge belonging to the genus Hymeniacidon (order Halichondrida) that yielded methanol-dichloromethane extracts active against the murine P388 lymphocytic leukemaia cell line. A 500 kg (wet weight) recollection of the organism was employed to isolate the cancer cell line inhibitory components utilizing the P388 cell line to guide the separations.

Recent examples include the nitrogen heterocyclic constituents diazonamide A (Li, J. et al., Total Synthesis of Nominal Diazonamides—Part 2: On the True Structure and Origin of Natural Isolates, Angew. Chem Int. Ed. 2001, 40, 4770-4773), bengamide B (Kinder, F. R. et al., Synthesis and Antitumor Activity of Ester-Modified Analogues of Bengamide B., J. Med. Chem. 2001, 44, 3692-3699), makaluvamine P (Casapullo, A. et al., Makaluvamine P, a New Cytotoxic Pyrroloiminoquinone from Zyzzya cf. fuliginosa, J. Natu. Prod. 2001, 64, 1354-1356), spongidepsin (Grassia, A. et al., Spongidepsin, a New Cytotoxic Macrolide from Spongia sp., Tetrahedron 2001, 57, 6257-6260), N-methyl-epi-manzamine D (Zhou, B-N., et al., New Cytotoxic Manzamine Alkaloids from a Palaun Sponge, Tetrahedron 2000, 56, 5781-5784), amphiasterin A3 (Zampella, A. et al., Amphiasterins: a New Family of Cytotoxic Metabolites from the Marine Sponge Plakortis quasiamphiaster, Tetrahedron 2001, 57, 257-263), and pyrinodemins B-D (Hirano, K. et al., Potent Cytotoxic bis-pyridine Alkaloids from Marine Sponge Amphimedon sp., Chem. Pharm. Bull. 2000, 48, 974-977).

The aaptamines form a small group of 1H-benzo[de][1,6]-naphthyridine marine alkaloids with cancer cell growth inhibitory properties. The parent aaptamine (1) was first isolated by Nakamura (Nakamura, H. et al., Isolation and Structure of Aaptamine, a Novel Heteroaromatic Substance Possessing alpha-blocking Activity from the Sea Sponge Aaptos aaptos, Tetrahedron Lett. 1982, 23, 5555-5558) and was found to possess antineoplastic as well as α-adrenoreceptor blocking activity (Ohizumi, Y., et al., Alpha-Adrenoceptor Blocking Action of Aaptamine, A Novel Marine Natural Product, in Vascular Smooth Muscle, J. Pharm. Pharmacol. 1984, 36, 785-786). The isolation of isoaaptamine (2) was first reported by Fedoreev from a sponge in the genus Suberites (Fedoreev, S. A., et al., Cytotoxic Activity of Aaptamines Derived from Suberitidae Sponges, Khimiko-Farmatsevticheskii Zhurnal 1988, 22, 943-946). Later, it was isolated by three other groups (Kashman, Y. et al., Recent Developments in Research on Metabolites from Red Sea Invertebrates, New J. Chem. 1990, 14, 729-740; Shen, Y-C., et al., Bioactive Constituents from Marine Sponge Aaptos aaptos, Taiwan Shuichan Xuehuikan 1997, 24, 117-125; Pettit, G. R., et al., Antineoplastic Agents 380. Isolation and X-ray Crystal Structure Determination of Isoaaptamine from the Republic of Singapore Hymeniacidon sp. and Conversion to the Prodrug Hystatin, J. Nat. Prod, not yet published).

Isoaaptamine (2) has been reported to be a PKC inhibitor (Patil, A. D., et al., Aaptamines as Protein Kinase C Inhibitors, PCT Int. App;. WO 95/0584, 1995), to possess activity against a number of clinically important microorganisms (Pettit, G. R., et al., Antineoplastic Agents 491. Synthetic Conversion of Aaptamine to Isoaaptamine and 9-demethylaaptamine, J. Org. Chem., not yet published) and to inhibit growth of cancer cells (Shen, Y-C., et al., Structures and Cytotoxicity Relationship of Isoaaptaime and Aaptamine Derivatives, J. Nat. Prod.,62, 1264-1267). In our laboratory, isoaaptamine (2) showed significant cytotoxicity against the murine P388 lymphocytic leukemia cell line (ED₅₀=0.28 μg/mL) and against a panel of six human cancer cell lines.

The first structure-activity relationship study of the aaptamines was summarized by Shen and coworkers. They observed that the phenolic group at the C-9 position was important for cytotoxicity. Acylation of that position led to a decrease in activity. (Shen, Y-C., et al., Structures and Cytotoxicity Relationship of Isoaaptamine and Aaptamine Derivatives, J. Nat. Prod., 1999, 62, 1264-1267.) Due to the interesting biological activities of isoaaptamine, the inventors began an extended chemical (SAR) and biological study of the aaptamines.

While aaptamine and isoaaptamine differ only in the position of one methyl group, its shift from the oxygen at the C-9 position in aaptamine to the N-1 position in isoaaptamine leads to an increase in cytotoxicity. The skeleton of aaptamine bears four positions which could be methylated, two nitrogen atoms and two phenol groups. Therefore, sixteen (16) different methyl or demethyl derivatives of the aaptamine scaffold are possible. The aim of the present investigation was the methylation and demethylation of aaptamine in order to explore the anticancer SAR possibilities. A parallel objective was to complete a practical methylation of aaptamine at N-1 and selective O-demethylation at the C-9 position to provide the much less abundant isoaaptamine in two steps.

Several total syntheses of aaptamine and a synthesis of isoaaptamine have been reported (Sugino, E., et al., Progress in Total Synthesis of Marine Alkaloids, Aaptamine, Heterocycles 1999, 50, 543-559; Walz, A. J., et al., Synthesis of 8-methoxy-1-methyl-1H-benzo[de][1,6]naphthyridin-9-ol(isoaaptamine) and Analogues, J. Org. Chem. 2000, 65, 8001-8010). Due to our multi-gram isolation of aaptamine from Hymeniacidon sp., this naphthyridine was employed as the starting material for our SAR studies (Pettit, G. R., et al., Antineoplastic Agents 380. Isolation and X-ray Crystal Structure Determination of Isaaptamine from the Republic of Singapore Hymeniacidon sp. and Conversion to the Prodrug Hystatin, J. Nat. Prod., not yet published).

Methylation of aaptamine (1) led to the formation of methylaaptamine (4). However, the methyl group proved to be bonded to the nitrogen atom at N-4, as shown in (4), and not as expected, at N-1. Further, methylation led to the formation of 1,4-dimethylaaptamine iodide 5, where both nitrogen-atoms N-1 and N-4 were methylated.

The 1H-benzo[de][1,6]-naphthyridine skeleton of aaptamine (1) consists of a quinoline as well as isoquinoline substructure. Interestingly, quaternary isoquinolinium or even quinolinium alkaloids are not unusual in nature and often have biological activity (Hallock, Y. F., et al., Gentrymine B, the First Quaternary Isoquinoline Alkaloid from Ancistrocladus korupensis, Tetrahedron Lett. 1995, 36, 4753-4756; Mitscher, L. A., et al., Antibiotics from Higher Plants: Pteleatinium Chloride, a New Quaternary Quinoline Alkaloid from Ptelea trifoliata with Antitubercular and Antiyeast Activity, J. Chem. Soc., Dalton Trans. 1971, 17, 1040-1040; Psotova, J., et al., Quaternary Isoquinoline Alkaloids as Protein Kinase C Inhibitors, Chemicke Listy, 1996, 90, 613-614; Del Poeta, M., et al., Comparison of In Vitro Activities of Camptothecin and Nitidine Derivatives Against Fungal and Cancer Cells, Antimicrob. Agents Chemother. 1999, 43, 2862-2868). For example, the alkaloid nitidine (6), from the roots of Toddalia asiatica, showed significant anti-HIV activity and inhibited the HIV reverse transcriptase, whereas the structurally related benzo[c]phenanthridine alkaloid NK109 showed anticancer activity (Tan, G. T., et al., Evaluation of Natural Products as Inhibitors of Human Immunodeficiency Virus Type 1 (HIV-1) Reverse Transcriptase, J. Nat. Prod. 1991, 54, 143-154; Yang, S. S., et al., Natural Product-Based Anti-HIV Drug Discovery and Development Facilitated by the NCI Developmental Therapeutics Program, J. Nat. Prod. 2001, 64, 265-277; Kanzawa, F., et al., Antitumor Activities of a New Benzo[c]phenanthridine Agent, 2,3-(methylenedioxy)-5-methyl-7-hydroxy-8-methoxybenzo[c]phenanthridinium Hydrogen Sulfate Dihydrate (NK109), Against Several Drug-Resistant Human Tumor Cell Lines, Br. J. Cancer 1997, 76, 571-581). Due to such considerations and the cancer cell growth inhibitory activity of the quaternary ammonium salt 5 (ED₅₀ 3.9 μg/mL, murine P388), we began a structural investigation of naphthyridine quaternary ammonium salts.

### SUMMARY OF THE INVENTION

Disclosed herein is the isolation and elucidation of the structure of isoaptamine (2), and its conversion to numerous novel related compounds as set forth below and which appear to have antimicrobial and/or cancer fighting properties. Also disclosed is the conversion of isoaaptamine (2) to the phosphate prodrug hystatin 1 (11), as well as the conversion of aaptamine to numerous compounds, including but not limited to:
9-demethyloxyaaptamine (3);
9-demethylaaptamine (18);
4-methylaaptamine (4);
1,4-dimethylaaptamine iodide (5);
4-N-methyl-8,9-dihydroxy-4H benzo[de][1,6]naphthyridine (7);
4-N-methyl-8 methoxy-9hydroxy-4H benzo[de][1,6]naphthyridine (8);
1-H-Benzo[de][1-6]naphthyridinium salts (9a-c);
hystatin 2 (10a); and
10b-d.

Set forth below are structural formulas for compounds of the invention:

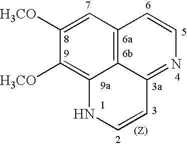

1, aaptamine

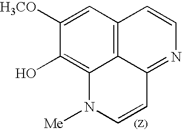

2, isoaaptamine

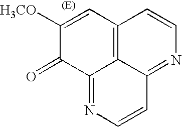

3, 9-demethyloxyaaptamine

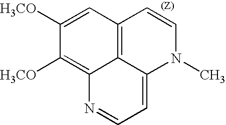

4, 4-N-metbylaaptamine

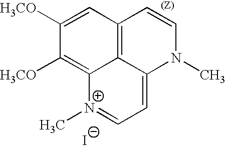

5, 1,4-dimethylaaptamine iodide

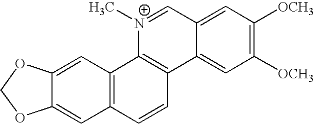

6, nitidine

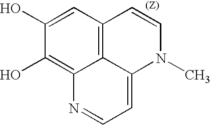

7

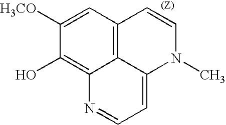

8

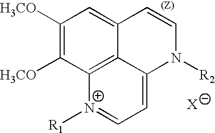

| | R₁ | R₂ | X |
|---|---|---|---|
| 9a | H | benzyl | Cl |
| b | H | 4-methoxylbenzyl | Cl |
| c | H | 3,4,5-trimethoxybenzyl | Cl |
| 10a | benzyl | benzyl | Cl |
| (hystatin | | | |
| 2) | | | |
| b | 4-methoxybenzyl | 4-methoxybenzyl | Br |
| c | 3,4,5-trimethoxybenzyl | 3,4,5-trimethoxybenzyl | Br |
| d | 3-hydroxy-4-methoxybenzyl | 3,4,5-trimethoxybenzyl | Br |

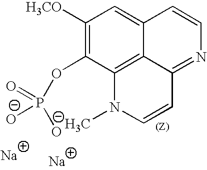

11, hystatin 1

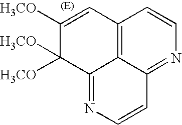

12

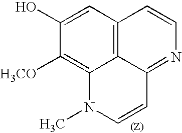

13

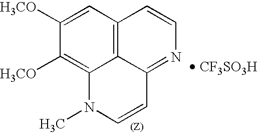

14

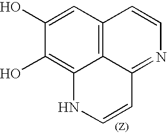

15

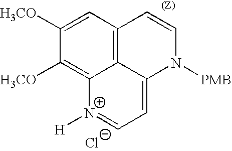

16

### DETAILED DESCRIPTION OF THE INVENTION RELATING TO ISOLATION AND STRUCTURAL ELUCIDATION OF ISOAAPTAMINE AND CONVERSION OF ISOAAPTAMINE TO HYSTATIN 1

The following describes the isolation and elucidation of the structure of isoaaptamine (2), and further describes a method for the conversion of isoaaptamine (2), which is relatively unstable, to its novel and more stable sodium phosphate prodrug, hystatin 1 (11).

Employing bioassay (murine P388 lymphocytic leukemia cell line) guided isolation procedures, extracts of the Republic of Singapore marine sponge Hymeniacidon sp. were found to contain demethyloxyaaptamine (3), and aaptamine (1), as prominent cancer cell growth inhibitory constituents accompanied by the trace, albeit more active component isoaaptamine (2). The isolation, X-ray structure elucidation, antineoplastic and antimicrobial activities of isoaaptamine (2) have been summarized. Because of instability, isoaaptamine (2) was converted to a stable sodium phosphate prodrug designated hystatin 1 (11).

In 1992, during the inventors' marine invertebrate survey in the Republic of Singapore, a previously unknown species of sponge belonging to the genus Hymeniacidon (order Halichondrida) was located that yielded methanol-dichloromethane extracts active against the murine P388 lymphocytic leukemia cell line. A 500 kg (wet wt) recollection of the organism was employed to isolate the cancer cell line inhibitory components utilizing the P388 cell line to guide the separations, as is described more fully below.

Results and Discussion

The sponge methanol-water and methanol-dichloromethane extracts (see Experimental Section) were partitioned between water and dichloromethane. The dichloromethane phase was partitioned between hexane and methanol-water (9:1) to give polar fraction A. The initial aqueous phase was next extracted with ethyl acetate followed by 1-butanol to yield alcohol soluble fraction B (1.08 kg). A series of Sephadex LH-20 gel permeation and partition chromatographic steps were used to separate fraction A and led to the previously known demethyloxyaaptamine (3) and the dimethyl ketal 12, likely an artifact of the isolation process (Nakamura, H., et al., J. Chem. Soc. Perkin Trans. 1 1987, 173-176). Demethyloxyaaptamine was isolated in gram quantities and exhibited a P388 ED₅₀ of 0.31 μg/mL, while the much less abundant dimethyl ketal 12 was inactive (P388 ED₅₀ 59 μg/mL). Fraction B was separated as summarized in Scheme 1.

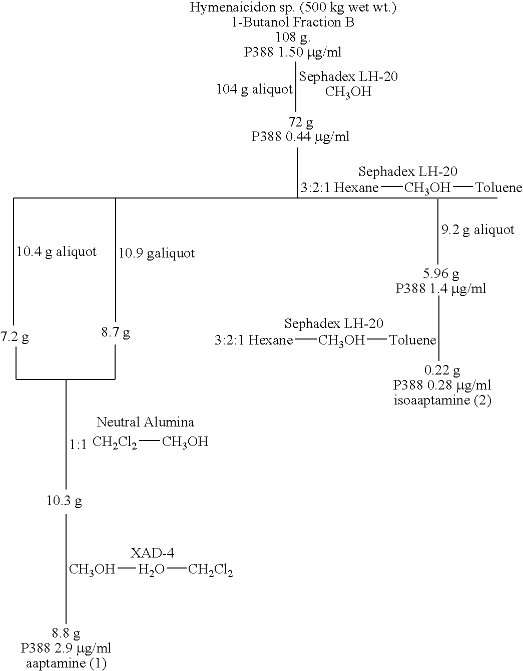

Aaptamine was also isolated in gram quantities along with the minor (10⁻⁵% yields) constituent isoaaptamine, both as the hydrochloride salt (Nakamura, H., et al., Tetrahedron Lett., 1982, 23, 5555-5558; Fedoreev, S. A., et al, Khim.-Farm. Zh. 1988, 22, 943-946; Kashman, Y., et al., New J. Chem. 1990, 14, 729-740; Shen, Y-C., et al., J. Fish. Soc. Taiwan, 1997, 24, 117-125; Shen, Y-C., et al., Taiwan Shuichan Xuehuikan 1997, 24, 117).

The cancer cell growth inhibitory activity of the compounds demethyloxyaaptamine (3), aaptamine (1), isoaaptamine (2) and hystatin 1 (11) are summarized in Table 1.

TABLE 1
Comparative Cancer Cell Line Results (ED₅₀ μg/mL)
for demethyloxyaaptamine (3), aaptamine (1), isoaaptamine
(2) and hystatin 1 (11)

| Cancer cell line | (3) | (1) | (2) | (11) |
|---|---|---|---|---|
| leukemia murine P388 | 0.31 | 3.60 | 0.28 | 3.0 |
| ovary OVCAR-3 | 0.39 | 4.90 | 1.20 | ND |
| CNS SF-295 | 2.80 | 4.10 | 2.60 | ND |
| renal A498 | 4.20 | 3.20 | 2.20 | ND |
| lung NCI-H460 | 2.30 | 3.20 | 2.40 | >10 |
| colon KM 20L2 | 2.10 | 3.60 | 2.30 | >10 |
| melanoma SK-MEL-5 | 1.00 | 4.30 | 1.60 | ND |

The structures of compounds demethyloxyaaptamine (3), its dimethyl ketal (12) and isoaaptamine (2) were readily deduced from spectral properties and literature comparisons (Nakamura, H., et al., J. Chem. Soc. Perkin Trans. 1 1987, 173-176; Nakamura, H., et al., Tetrahedron Lett., 1982, 23, 5555-5558). However, the structure of isoaaptamine 2 proved to be troublesome. Analysis of the ¹H-and ¹³C-NMR spectra indicated that it was nearly identical to aaptamine which was first reported by Fedoreev (Fedoreev, S. A., et al, Khim.-Farm. Zh. 1988, 22, 943-946). Subsequently, it was again isolated by Kashman and Shen (Kashman, Y., et al., New J. Chem. 1990, 14, 729-740; Shen, Y-C., et al., J. Fish. Soc. Taiwan, 1997, 24, 117-125). In the inventors' experiments, all of the proton and carbon signals were shifted downfield slightly owing to its isolation as the hydrochloride salt. The shift of one of the methyl carbon resonances from *60.7 in aaptamine to 45.9 in isoaaptamine suggested the likelihood that an O-methyl had shifted to an N-methyl position. Since isoquinoline structures 8 and 13 also seemed consistent with the NMR data, it was apparent that structure 2 could not initially be assigned with certainty. But scrutiny of the NMR NOESY and ROESY spectra did point to the likelihood of structure 2. As an example, a cross peak in the ROESY spectrum between H-7 (*7.15; and the pyridine-like hydrogen H-6 (*7.15) was consistent with structure 2. NOESY cross peaks between the proposed methoxy on C-8 (*4.03) and both H-7 and the phenolic hydroxy (*9.4) were commensurate with structures 2 or 8 and allowed us to rule out the methoxy analogue 13. On the basis of the NMR data, we were unable to rule out the possibility of structure 8 unambiguously. Because of possible long range correlations which can occur through azaheterocycles, and in consideration of the important cancer cell line and antimicrobial activity of isoaaptamine (2), approaches to an unequivocal structure elucidation were undertaken based on X-ray crystal structure determination.

Since compound 2 was isolated as a yellow amorphous hydrochloride salt that resisted crystallization, other salt derivatives were explored. Changing the anion to perchlorate, for example, gave beautiful yellow-red needles. Unfortunately, these proved to be too thin for X-ray analysis. Other anions which were evaluated were even less promising. In view of the electron-rich structure of 2, a picric acid derivative was considered, since it might crystallize as both an acid-base and Π-Π complex. This proved to be the case. Single crystals of the picrate were formed by slow crystallization of a methanol-toluene solution of picric acid and isoaaptamine (2). More recently, isoaaptamine (2) has been synthesized by Walz and Sundberg (Walz, A. J., et al., J. Org, Chem. 2000, 65, 8001-8010).

For a variety of reasons, the isolation of isoaaptamine (2) as the most interesting cancer cell growth inhibitor of the Singapore sponge Hymeniacidon sp. is noteworthy. Aaptamine (1), as the parent alkaloid, was first isolated from the marine sponge Aaptos aaptos (Nakamura, H. et al., Tetrahedron Lett. 1982, 23, 5555-5558). Later, it was isolated-from several other species of sponges (Berquist, P. R., et al., Biochem. Syst. Ecol. 1991, 19, 289-290). The discovery of the aaptamines in Hymeniacidon sp. suggests that unusual isoquinolines may enjoy a broader species and geographic distribution. Also, the large quantities of aaptamine (1) contained in Hymeniacidon sp. would suggest a relatively important biological role. Consequently, the inventors proceeded with an extended study of the aaptamines, especially isoaaptamine (2).

When newly isolated, isoaaptamine (2) appears as a yellow colored powder that rapidly changes to dark green and finally brown or black, presumably due to air oxidation. Because of this instability and resultant degradation, attempts at preparation of a variety of simple derivatives generally proved to be elusive. Hence, attention was directed at preparation of a stable prodrug that would retain the biological activity. For that purpose, isoaaptamine (2) was first phosphorylated with dibenzylphosphate (Pettit, G. R. et al., Anti-Cancer Drug Design 2000, 15, 203-216; Silverberg, L. J., Tetrahedron Lett. 1996, 37, 771-774). Cleavage of the benzyl ester by means of trimethylsilyl bromide (Pettit, G. R. et al., Anti-Cancer Drug Design 1998, 13, 981-993; Lazar, S., Synth. Commun. 1992, 22, 923-931) and reaction of the resulting phosphoric acid with sodium methoxide afforded the relatively more stable disodium phosphate prodrug designated hystatin 1 (11). Scheme 2 below illustrates the aforementioned synthesis method. 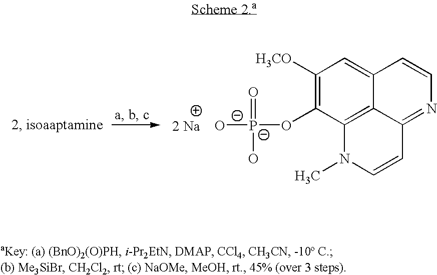
Biology

Evaluation of demethyloxyaaptamine (3), aaptamine (1), isoaaptamine (2) and hystatin 1 (11) against the Arizona State University Cancer Research Institute's human tumor cell line panel (Table 1) and the NCI 60-cell line human tumor screen were undertaken. (Boyd, M. R., Cancer: Principles and Practices of Oncology Updates; DeVita, et al., Eds; Lippincott: Philadelphia, 1989, Vol. 10, No. 3, pp. 1-12; Boyd, et al., Drug Development Research 1995, 34, 91-109; Boyd, M. R., et al. Cytotoxic Anticancer Drugs: Models and Concepts for Drug Discovery and Development; Valeriote, A., et al., Eds, Kluwer Academic Publishers: Amsterdam, 1992, pp. 11-34.)

Isoaaptamine (2) had antifungal and antibacterial activities, and the prodrug hystatin 1 (11) retained some of the antibacterial activity (Table 2).

TABLE 2
Comparative antimicrobial activities of isoaaptamine
(2) and hystatin 1 (11)

Range
of minimum
inhibitory
concentration
(μg/ml)

| Microorganism | (2) | (11) |
|---|---|---|
| Cryptococcus neoformans | 64 | * |
| Candida albicans | * | * |
| Staphylococcus aureus | 16-32 | 64 |
| Streptococcus pneumoniae | 8-32 | 16-32 |
| Enterococcus faecalis | 32-64 | * |
| Micrococcus luteus | 32-64 | * |
| Escherichia coli | * | * |
| Enterobacter cloacae | * | * |
| Stenotrophomonas maltophilia | * | * |
| Neisseria gonorrhoeae | <0.5 | <0.5 |

* no inhibition at 64 ug/ml

Experimental Section

General Experimental Procedures. Solvents used for chromatographic procedures were redistilled. Sephadex LH-20 (25-100 m) employed for gel permeation and partition chromatography was obtained from Pharmacia Fine Chemicals AB, Uppsala, Sweden. Gilson FC-220 and FC-202 fraction collectors were used for chromatographic fractionation experiments. Silica gel GF Uniplates were employed for monitoring the chromatographic separations. All TLC plates were visualized under UV light (254 nm) and developed with phosphomolybdic acid spray reagent. Uncorrected melting points were determined on a digital Electrothermal apparatus. The ¹H-, ¹³C- and ¹⁹FNMR spectra experiments were performed with a Varian VXR-500 or a VXR 400 instrument in the indicated solvent. Mass spectral data were recorded using a Varian MAT 312 instrument (EIMS), and IR spectra were determined with a Mattson Instruments 2020 Galaxy Series FTIR instrument. The X-ray crystal structure data collections were performed on an Enraf-Nonius CAD4 diffractometer or a Bruker SMART 6000 diffractometer. High resolution mass spectra were obtained on a JEOL LCMate magnetic sector instrument in the APCI mode with polyethylene glycol as reference or by FAB with a glycerol matrix.

Hymeniacidon sp. (Halichondrida:Hymeniacidonidae). The initial specimen of this bright orange encrusting-lobular sponge, a previously undescribed species, was initially collected on the north side of Terumbu Pemalang Besar Reef, Republic of Singapore at a depth of 5 to 10 meters in 1989. During 1992, 500 kg (wet wt) of the sponge was recollected in this general area.

Animal Extraction and Solvent Partitioning. The methanol shipping solution was removed and extract concentrated to 100 L of an aqueous suspension which was successively extracted with dichloromethane (6×50 L), ethyl acetate (6×50 L) and 1-butanol (4×50 L). The 1-butanol fraction was concentrated to dryness and partially redissolved in 2 L of methanol.

Isolation of the Aaptamines. The methanol soluble portion (108 g) of the 1-butanol fraction was fractionated by a gel permeation/partition sequence as outlined in Scheme 1. The major component isolated was aaptamine (1, 8.8 g, 1.7×10⁻³%) having spectral properties in complete accord with the literature values. The minor but more active isoaaptamine (2, 0.22 g, 4.4×10⁻⁵%) was obtained as an amorphous yellow powder; mp 200-205° C. (dec); ¹H-NMR (DMSO) 3.96 (s, 3H, OMe), 4.03 (s, 3H, NMe), 6.25 (d, J=7.2, 1 H, H-3), 6.80 (d, J=7.3, 1 H, H-6), 7.15 (s, 1 H, H-7), 7.25 (d, J=7.3, 1 H, H-5), 7.73 (d, J=7.2, 1 H, H-2), 9.4 (br s, 1 H, OH), 12.7 (br s, 1 H, NH); ¹³C-NMR (DMSO) 45.9 (NMe), 56.5 (OMe), 97.3 (C-3), 101.5 (C-7), 113.1 (C-6), 118.1 (C-9b), 127.7 (C-5), 129.3 (C-6a and C-9a), 132.2 (C-9), 148.9 (C-2), 149.2 (C-3a), 153.6 (C-8); MS m/z 228 (100), 213 (94), 185 (60), 170 (47), 155 (10), 142 (22), 127 (7), 115 (12), 84 (7). HREIMS m/z 228.0903 (calcd for C₁₃H₁₂N₂O₂: 228.0899).

Isoaaptamine Perchlorate. A sample of isoaaptamine (2) (1.0 mg) was dissolved in methanol (1.0 mL) in a small crystallization tube and two drops of perchloric acid (70%) allowed to run down the side of the tube. Thin red needles were slowly deposited after standing at room temperature for several hours. The crystals proved to be too thin for X-ray analysis and were not further characterized due to decomposition on standing.

Isoaaptamine picrate. Picric acid was washed with water and the damp acid was dissolved in methanol at a concentration of about 1 M. To a 1.0 mg sample of isoaaptamine (2) hydrochloride in a few drops of methanol was added 0.25 mL of the picric acid solution. The orange solution was heated to about 60° C. and diluted to 1.0 mL with toluene. The solution was filtered hot, allowed to cool to room temperature and then placed in a freezer (approx. −20° C.) where red colored single crystals (d.p. 95-97°) slowly formed over 72 hours.

X-Ray Crystal Structure Determination of Isoaaptamine (2). A few, marginal quality, red, rod-shaped crystals of the complex were obtained from toluene-methanol solution. The best specimen, 0.48×0.10×0.04 mm, was mounted on the tip of a glass fiber with Superglue. Data collection was performed at 24±1° for a monoclinic system. All reflections corresponding to a complete quadrant, with 20Θ130°, were measured using the ω/2Θ) scan technique. Subsequent statistical analysis of the complete reflection data set using the XPREP program, indicated the space group was P2₁/c, the asymmetric unit of the cell containing a single molecule each of the parent heterocyclic compound and picric acid, along with ½ a molecule of toluene. Crystal data: C₁₃H₁₂N₂O₂.C₆H₃N₃H_{7 .}½ C₇H₈, monoclinic space group P2₁/c, with a=7.983(1), b=12.839(3), c=21.522 (5) Å, β=92.920(1)°, V=2203.0(8) Å³, 8 (Cu Kα)=1.54184 Å, ρ_{c}=1.518 g cm⁻³ for Z=4 and F.W.=503.43, F(000)=1044. After Lorentz and polarization corrections, merging of equivalent reflections and rejection of systematic absences, 3141 unique reflections (R(int)=0.0505) remained, of which 2353 were considered observed (Iₒ>2σ(Iₒ)) and were used in the subsequent structure determination and refinement. Linear and anisotropic decay corrections were applied to the intensity data as well as an empirical absorption correction (based on a series of psi-scans) (North, A. C., et al., Acta. Crysta. 1968, A24, 351-359.)

Structure determination was readily accomplished with the direct-methods program SIR92 (Altomare, A., et al., SIR92-A Program for Automatic Solution of Crystal Structures by Direct Methods, Dipartimento Geomineralogico, Univ. of Bari, Italy.) All non-hydrogen atom coordinates (excluding toluene solvent atoms) were located in a structure solution run using the RANDOM option and allowing NREF to have the maximum value (499) for that program. The non-hydrogen solvent and the hydrogen atom positions on isoaaptamine (2) and picric acid were determined from difference Fourier maps using the program SHELXL-93 (Sheldrick, G. M., SHELXL93, Program for the Refinement of Crystal Structures, Univ. of Guttingen, Germany 1993). The hydrogen atoms were assigned thermal parameters equal to 1.5 the Uiso value of the atom to which they were attached and then both coordinates and thermal values were forced to ride that atom during final cycles of refinement. All non-hydrogen atoms were refined anisotropically in a full-matrix least-squares refinement process with the SHELXL-93 software package. The assignment of nitrogen atom positions in the aromatic ring of the heterocycle could not be made with any certainty based upon bond distance information alone. Consequently, N vs C occupancy refinements were made on the individual models produced by alternating the N atom over all possible suspected positions which might contain nitrogen. The N and C character of the various positions investigated were calculated to be the following: 1, 77% N, 23% C; 2, 31% N, 69% C; 3, 15% N, 85% C; 3A, 0% N, 100% C; 4, 97% N, 3% C; 5, 15% N, 85% C;, 6, 38% N, 62% C. As a result, the two nitrogen atoms were assigned to the 1 and 4 positions, respectively. The final standard residual R value for the solvated complex model was 0.0769 for observed data (2353 reflections) and 0.0985 for all data (3141 reflections). The corresponding Sheldrick R values were wR₂ of 0.2094 and 0.2269, respectively. A final difference Fourier map showed insignificant residual electron density; the largest difference peak and hole being 0.432 and −0.326 e/Å³, respectively. Final bond distances and angles were all within acceptable limits.

Synthesis of Hystatin 1 (Disodium isoaaptamine 9-O-phosphate) (11). To a dry flask equipped with a septum, magnetic stirrer, thermometer and Ar inlet containing dry acetonitrile (20 mL) was added isoaaptamine hydrochloride (2, 0.26 g, 0.97 mmol). Upon cooling to −10° C., tetrachloromethane (0.47 mL, 4.85 mmol) was added followed by diisopropylethylamine (0.51 mL, 2.91 mmol) and a catalytic amount of dimethylaminopyridine. After 2 minutes, dropwise addition of dibenzyl phosphate (0.30 mL, 1.36 mmol) was begun while maintaining a temperature of −10° C. The resultant yellow solution was stirred for 2 hours and 0.5 M aqueous KH₂PO₄ (30 mL) was added. The water layer was extracted with CH₂Cl₂ (4×50 mL) and the combined organic extract was washed with brine (100 mL). The organic layer was dried and the solvent was removed in vacuo. Purification by column chromatography (neutral alumina, CH₂Cl₂—CH₃OH, 20:1 as eluent) gave the dibenzyl phosphate as a yellow oil (0.27 g) which was immediately dissolved in dichloromethane. Bromotrimethylsilane (0.2 mL) was added at room temperature. After stirring for 2 hours, the solvent was removed and the residue was dissolved in water (20 mL). The water layer was washed with dichloromethane (3×10 mL) and the water was removed. The yellow solid was dissolved in methanol, a 30% solution of sodium methoxide (0.22 mL, 1.16 mmol) was added and the mixture was stirred for 12 hours. The reaction mixture was concentrated to a yellow solid that was recrystallized from acetone-water. The isoaaptamine prodrug (11) was obtained as a yellow-green powder (0.15 g, 45% yield): mp 166-168° C. (dec.); UV (CH₃OH) 8ₘₐₓ (log ε) 209 nm (4.22), 241 (4.17), 260 (4.28), 316 (3.49), 326 (3.39), 389 (3.78); IR (KBr): v 3406 (s), 3227 (m), 1649 (s), 1604 (s), 1525 (w), 1465 (m), 1429 (w), 1342 (m), 1298 (m), 1240 (w), 1111 (s), 977 (s), 848 (m), 723 (m), 630 (m), 563 (m); ¹H NMR (D₂O and a few drops of CD₃OD): *3.77 (s, 3H, NMe), 3.87 (s, 3H, OMe), 5.84 (d, J=7.5 Hz, 1H, H-3), 6.47 (d, J=7.5 Hz, 1H, H-6), 6.62 (d, J=7.5 Hz, 1H, H-5), 6.65 (s, 1H, H-7), 7.43 (d, J=7.5 Hz, 1H, H-2); ¹³C NMR (D₂O): *=45.81, 56.32, 98.05, 101.49, 112.90, 117.34, 128.96, 132.80, 134.10, 147.76, 148.32, 158.62; ³¹P NMR (162 MHz, D₂O) *1.58; EIMS m/z 228 (100) [M⁺-(NaO)₂P(O)H], 213 (58), 185 (39), 170 (24), 142 (13), 114 (12), 28 (31); HRFABMS ([M-Na₂O₃P+H] m/z 229.09392 (cald for C₁₃H₁₃N₂O₂, 229.09771).

Antimicrobial Susceptibility Testing. Compounds were screened against the bacteria Staphylococcus aureus, Streptococcus pneumoniae, Enterococcus faecalis, Micrococcus luteus, Escherichia coli, Enterobacter cloacae, Stenotrophomonas maltophilia and Neisseria gonorrhoeae, and the fungi Candida albicans and Cryptococcus neoformans, according to established broth microdilution susceptibility assays (National Committee for Clinical Laboratory Standards, Methods for Dilution Antimicrobial Susceptability Tests for Bacteria that Grow Aerobically, Approved Standard M7-a4, Wayne, Pa.: NCCLS, 1997; National Committee for Clinincal Laboratory Standards, Reference Method for Broth Dilution Antifungal Susceptibility Testing of Yeasts, Approved Standard M27-A, Wayne, Pa.: NCCLS, 1997). The minimum inhibitory concentration was defined as the lowest concentration of compound that inhibited all visible growth of the test organism (optically clear). Assays were repeated on separate days. Results of this testing are shown in Table 2 above. Mycobacterium tuberculosis H₃₇Rv was screened using the Microplate Alamar Blue Assay. (Collins, L. et al., Microplate Alamar Blue Assay Versus BACTEC 460 System for High-throughput Screening of Compounds against Mycobacterium tuberculosis and Mycobacterium avium, Antimicrob. Agents Chemother. 1997, 41, 1004-1009).

### DETAILED DESCRIPTION OF THE INVENTION RELATING TO THE CONVERSION OF AAPTAMINE TO ISOAAPTAMINE, 9-DEMETHYLAAPTAMINE AND 4-METHYLAAPTAMINE

Aaptamine (1) was used as starting material for synthetic transformation to isoaaptamine (2), 9-demethylaaptamine (18), and 4-methylaaptamine (4). A general method for the selective O-demethylation of such 1H-benzo[de][1,6]-naphthyridine (1) marine sponge constituents at position C-9 has been developed. Selective O-demethylation of aaptamine (1) and 1-methylaaptamine (14) with 48% hydrobromic acid led to 9-demethylaaptamine (18) and isoaaptamine (2) respectively. A selection of other aaptamine derivatives were synthesized and their structures were unambiguously determined by X-ray methods. In addition, their cancer cell growth inhibitory properties were evaluated against the murine P388 lymphocytic cell line and a mini-panel of human cancer cell lines. Evaluation as inhibitors of the PKC signal transduction pathway and against a selection of microorganisms was also undertaken. Aaptamine derivatives 15 and 18 had broad-spectrum antimicrobial activities.

The first structure-activity relationship study of the aaptamines was summarized by Shen and coworkers (Shen, Y. C., et al., J. Nat. Prod., 1999, 62, 1264-1267). They observed that the phenolic group at the C-9 position of the aaptamines has been observed previously as important for cytotoxicity. Acylation of that position led to a decrease in activity. While aaptamine (1) and isoaaptamine differ only in the position of one methyl group, its shift from the C-9 position in aaptamine (1) to the N-1 position in isoaaptamine (2) leads to an increase in cytotoxicity. The skeleton of aaptamine bears four positions which could be methylated, two nitrogen atoms and two phenol groups. Therefore, 16 different methyl or demethyl derivatives of the aaptamine scaffold are possible.

The aim of the present investigation was the methylation and demethylation of aaptamine (1) in order to explore the anticancer SAR possibilities. A parallel objective was to complete a practical methylation of aaptamine (1) at N-1 and selective O-demethylation at the C-9 position to provide the much less abundant isoaaptamine (2) in two steps. Due to the inventors' multi-gram isolation of aaptamine (1) from Hymeniacidon sp., this marine sponge was employed as the starting material (Pettit, G. R. et al., J. Nat. Prod., not yet published).

Results and Discussion

The key step in our synthesis of isoaaptamine (2) from aaptamine (1) involved selective O-demethylation at the C-9 position. Several methods are known for O-demethylation of isoquinoline derivatives. For example, Alvarez used boron tribromide in dichloromethane as part of a synthesis of batzelline B (Alvarez, et al., Eur. J. Org. Chem. 1999, 5, 1173) and Pelletier employed hydrobromic acid for the selective O-demethylation of 6,7-dimethoxy-3,4-dihydroisoquinoline (Pelletier, J. C., et al., J. Org. Chem. 1987, 52, 616). Because of the limited solubility of aaptamine (1) in dichloromethane, we used the latter method in a number of experiments under modified conditions. Treatment of aaptamine (1) with 48% hydrobromic acid at 145-150 ° C. yielded only diphenol 15, whereas at 115-120 ° C., aaptamine (1) was selectively O-demethylated at the C-9 position (Scheme 3). The NMR data of 9-demethylaaptamine (18) were in good agreement with those reported by Nakamura et al. (Nakamura, H., et al., J. Chem. Soc. Perkin Trans. 1 1987, 173). The structure of monophenol 18 was confirmed as the hydrobromide trihydrate via X-ray methods.

Upon successful development of the selective O-demethylation, we began N-methylation studies of aaptamine (1). Reaction of aaptamine (1) with excess methyl iodide and potassium carbonate led to formation of the permethylated quaternary ammonium salt 5, the structure of which was again established via X-ray methods. Two new singlets appeared in the ¹H NMR which confirmed methylation of N-1 and N-4. Such quaternary isoquinolinium alkaloids are not unusual in nature and they often show interesting biological activities.

The selective methylation of aaptamine (1) was eventually achieved using sodium hexamethyldisilazane (NaHMDS) and methyl iodide in THF at −78 ° C. (Scheme 3). However, the methyl group proved to be bonded to the nitrogen atom N-4 and not as expected to N-1. The X-ray crystal structure of 4-methylaaptamine (4) confirmed that the methyl group was attached to nitrogen N-4. Our ¹H and ¹³C NMR data of 4-methylaaptamine (4) were in good agreement with those reported by Coutinho et al. (Coutinho, A. F., Heterocycles 2002, 57, 1265). Treatment of 4-methylaaptamine (4) with 48% HBr at 145-150 ° C. led to the formation of diphenol 7. Selective O-demethylation of dimethyl ether 4 at position C-9 was achieved with 48% HBr at 115° C. and led to a structural isomer (8) of isoaaptamine (2). With monophenols (2 and 8) differing only in the position of the methyl group, the ¹H NMR spectra were very helpful. The singlet for the N-methyl group of isoaaptamine (2) appears at 4.03 ppm and that of isomer 8 at 3.56 ppm. While this result was interesting and potentially useful, a new strategy for the synthesis of isoaaptamine (2) needed to be devised, as is set forth below in Scheme 3.

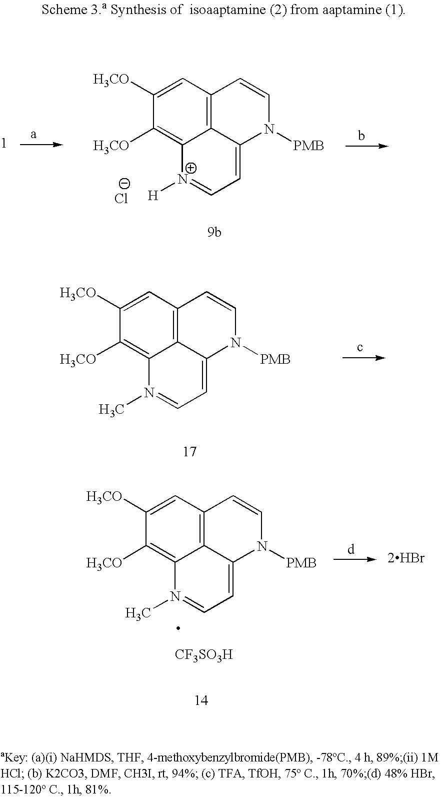

After a number of unsuccessful approaches, treatment of aaptamine (1) with NaHMDS and 4-methoxybenzyl bromide at −78° C. led to the PMB-protected aaptamine 16. Reaction of the PMB-derivative with potassium carbonate and excess methyl iodide gave the desired N-1 methylated product (17). The position of the methyl-group was unambiguously demonstrated by an X-ray structure determination. The X-ray crystal structures of aaptamine derivatives 16 and 17 confirmed that the methyl group was attached to the quaternary nitrogen N-1.

Surprisingly, cleavage of the PMB-protecting group proved to be difficult. No reaction was observed using TFA at 70° C. With ceric ammonium nitrate or 48% hydrobromic acid, only decomposition was observed. Finally, treatment of PMB derivative 17 with a mixture of TFA and trifluoromethane-sulfonic acid led to removal of the PMB group. The quartet in the ¹³C NMR at 121.8 ppm and the singlet in the ¹⁹F NMR at 44.1 ppm confirmed the formation of 1-methyl-aaptamine (14) as a trifluoromethane-sulfonic acid salt. Trimethyl derivative 14 was also prepared by Hibino and Sundberg (Hibino, S., et al., J. Chem. Soc., Perkin Trans 1 1998, 2429; Walz, A. J., J. Org. Chem. 2000, 65, 8001). The selective O-demethylation of dimethyl ester 14 with 48% HBr at 115° C. gave the natural product isoaaptamine (2) as the hydrobromide salt. Both ¹H and ¹³C NMR spectral comparison of synthetic isoaaptamine (2) with a sample of natural origin confirmed that they were identical.

Biology The cancer cell growth inhibitory properties were examined using the murine P388 lymphocytic leukemia cell line and a selection of human cancer cell lines. The results are summarized in Table 3.

TABLE 3
Cancer cell growth inhibitory (ED₅₀ μg/ml) activity of isoaaptamine
(2), 9-demethylaaptamine (18) and several other aaptamine-derivatives.

| | Compound | | | | | | | | | |
|---|---|---|---|---|---|---|---|---|---|---|
| Cancer cell line | (2) | (15) | (5) | (18) | (4) | (7) | (8) | (16) | (11) | (17) |
| leukemia P-388 | 1.6 | 0.12 | 3.9 | 0.22 | >10 | 2.2 | >10 | 2.2 | 0.23 | 3.1 |
| pancreas BXPC-3 | 4.1 | 0.33 | 7.1 | 1.6 | >10 | >10 | >10 | 4.2 | 0.74 | 7.0 |
| breast MCF-7 | 2.2 | 0.31 | 4.9 | 0.66 | >10 | 4.1 | >10 | 8.0 | 2.2 | 8.0 |
| CNS SF-268 | 2.2 | 0.80 | 0.69 | 1.4 | >10 | 5.6 | >10 | 8.2 | 0.28 | 5.9 |
| lung NCI-H460 | 2.9 | 0.52 | >10 | 2.0 | >10 | >10 | >10 | 5.1 | 1.9 | 4.6 |
| colon KM 20L2 | >10 | 0.3 | 6.1 | 1.7 | >10 | >10 | >10 | 3.9 | 0.88 | 5.4 |
| prostrate DU-145 | 3.3 | 1.1 | 3.5 | 1.8 | >10 | >10 | >10 | 3.1 | 0.52 | 7.5 |

In general, O-demethylation led to an increase in inhibitory activity. For example, 9-demethylaaptamine (18) and diphenol 15 were more inhibitory than aaptamine (1), and isoaaptamine (2) exhibited greater activity than the methylaaptamine 14. Inhibitory activity was markedly reduced or eliminated by methylation of aaptamine (1). Methylation at N-4 led to inactive derivatives (4 and 8). Importantly, 4-methylaaptamine (4) showed potent antiviral activity against herpes simplex virus type 1 (HSV-1) and low toxicity to Vero cells. Only diphenol 7 and methylaaptamine 14 showed even marginal activity. Interestingly, the quaternary ammonium salts 5 and 17 exhibited significant inhibitory activity against the murine P388 lymphocytic leukemia and the human cancer cell lines.

Isoaaptamine (2) and derivatives 4, 5, 7, 8, and 14-18 were evaluated as ligands for PKC, based on the initial report of activity for isoaaptamine (Patil, et al., PCT Int. Appl. WO 95/0584 March 1995). Activity was measured at 30 μM compound using inhibition of [³H-20]phorbol 12,13-dibutyrate binding to PKC alpha as described previously (Kazanietz, M. G., et al., Molecular Pharmacol., 1993, 44, 298). Assays were carried out with triplicate measurements in either single or triplicate experiments, depending on the compound. Only 13% inhibition was observed for 2 and less than 10% inhibition for the other derivatives. We conclude that these compounds do not show appreciable affinity for PKC.

The series of compounds 4, 5, 7, 8, and 14-18 described here were examined for possible effects on tubulin polymerization, under conditions in which the colchicine site agent combretastatin A-4 yielded an IC₅₀ value of about 2 μM for inhibition of extent of assembly. Only minimal inhibitory activity was observed. While compounds 15 and 8 did inhibit the rate of polymerization by over 50% at 40 μM (the highest concentration examined), an IC₅₀ value for inhibition of assembly extent was obtained only for compound 18. The IC₅₀ for compound 18 was 37±4 (SD) μM, about 18-fold higher than the value obtained for combretastatin A-4. Thus, it seems unlikely that the cytotoxic mechanism for these aaptamine analogs derives from an interaction with tubulin.

9-Demethylaaptamine (18) is known to have activity against Gram-positive and Gram-negative bacteria (Nakamura, H., et al., J. Chem. Soc., Perkin Trans. 1 1987, 173). As shown in Table 4, the inventors determined that phenol 18 also has antifungal activity, and that the new aaptamine derivative 15 had antibacterial and antifungal activities.

TABLE 4
Comparative Antimicrobial activities of aaptamine derivatives

| | Range of minimum inhibitory concentration (μg/ml) | | | | | | | | |
|---|---|---|---|---|---|---|---|---|---|
| Microorganism | 15 | 5 | 18 | 4 | 7 | 8 | 16 | 17 | 14 |
| Cryptococcus neoformans | 32 | * | 32 | * | * | * | * | * | * |
| Candida albicans | 64 | * | 64 | * | * | * | * | * | * |
| Staphylococcus aureus | 4-8 | * | 16 | * | * | * | * | * | * |
| Streptococcus pneumoniae | 8 | * | 2-4 | * | * | * | 64 | * | 64 |
| Enterococcus faecalis | 8-16 | * | 8-32 | * | * | * | * | * | * |
| Micrococcus luteus | 4 | 16-32 | 8 | * | * | * | 64 | 4-8 | * |
| Escherichia coli | 16-64 | * | 32-64 | * | * | * | * | * | * |
| Enterobacter cloacae | * | * | * | * | * | * | * | * | * |
| Stenotrophomonas maltophil | 32 | * | * | * | * | * | * | * | * |
| Neisseria gonorrhoeae | >0.5 | * | >0.5 | * | 64 | 4 | 64 | 64 | * |

*no inhibition at 64 μg/ml

The antimicrobial activity of phenols 15 and 18 in the presence of human serum was also investigated. For both compounds, minimum inhibitory concentrations were identical or one 2-fold dilution lower than those obtained in the absence of serum.

Thus, the inventors have developed methods for the convenient syntheses of isoaaptamine (2), 9-demethylaaptamine (18) and a selection of methyl derivatives have been developed. With the procedures described herein, 8 of the 16 possible O and N methyl-derivatives of aaptamine were synthesized. A selection of these naphthyridines were found to exhibit significant cancer cell growth and antimicrobial inhibitory activities. They did not function, however, as ligands for PKC, despite earlier reports (Patil, A. D. et al., PCT Int. Appl. WO 95/0584 March 1995).

Experimental Section

General Experimental Procedures.

### 8,9-Dihydroxy-1H-Benzo[de][1,6]naphthyridine hydrobromide (15)

Aaptamine (1) hydrochloride (0.50 g, 1.89 mmol) was dissolved in 48% HBr (5 mL) and the solution stirred at 145-150° C. (preheated oil-bath). After 4 hours the mixture was cooled to room temperature. The solution was filtered, and the solid was washed with ethyl acetate to give diphenol 15 as a green powder (0.41 g, 77%). Further purification was achieved by column chromatography on Sephadex LH-20 with CH₃OH as eluent: mp 245-248° C. (dec); UV (CH₃OH) 8ₘₐₓ (log ε) 205 (3.93), 245 (4.08), 269 (3.96), 306 (3.31), 360 (3.33), 4.08 (3.27) nm; IR (KBr)ₘₐₓ 1655, 1626, 1556, 1444 cm⁻¹; ¹H NMR (500 MHZ, CD₃OD) *6.16 (d, J=7.5 Hz, 1H, H-3), 6.61 (d, J=7.3 Hz, 1H, H-6), 6.73 (s, 1H, H-7), 6.98 (d, J=7.3 Hz, 1H, H-5), 7.63 (d, J=7.5 Hz, 1H, H-2); ¹³C NMR (126 MHZ, CD₃OD) *98.1, 105.3, 113.9, 117.7, 128.1, 129.8, 130.9 (2C), 142.2, 151.6, 152.4; and EIMS m/z 200 (100) [M⁺], 171 (42), 154 (15), 82 (25), 80 (26), 28 (64); HRMS [APCI⁺] m/z [M-Br]⁺201.0670 (calcd for C₁₁H₉N₂O₂, 201.0664).

1,4-Dimethyl-8,9-dimethoxy-4H-benzo-[de][1,6]naphthyridin-1-ium iodide (5). Part A. Aaptamine (1) hydrochloride (0.50 g, 1.89 mmol) was dissolved in anhydrous dimethylformamide (50 mL). Potassium carbonate (1.31 g, 9.45 mmol) and methyl iodide (0.56 mL, 9.45 mmol) were added at room temperature. After stirring for 12 hours at the same temperature, the solution was filtered, and the solvent was removed in vacuo to leave a brown oil. The residue was purified by column chromatography (silica gel, CH₂Cl₂—CH₃OH 6:1) to give the product as a bright yellow solid (0.60 g, 82%). Yellow needles were obtained by crystallization from CH₂Cl₂—CH₃OH: mp 215-217° C. (dec); R_{f} 0.53 (CH₂CL₂—CH₃OH 10:1); UV (CH₃OH) 8ₘₐₓ (log ε) 208 (4.39), 220 (4.45), 241 (4.38), 261 (4.39), 271 (4.38), 279 (4.35), 397 (3.84) nm; IR (KBr)ₘₐₓ 1647, 1570, 1350, 1305, 1084 cm⁻¹; ¹H NMR (500 MHZ, CD₃OD) *3.63 (s, 3H, CH₃), 3.77 (s, 3H, CH₃), 3.98 (s, 3H, CH₃), 4.04 (s, 3H, CH₃), 6.49 (d, J=7.8 Hz, 1H, H-3), 6.91 (d, J=7.3 Hz, 1H, H-6), 7.25 (s, 1H, H-7), 7.54 (d, J=7.3 Hz, 1H, H-5), 8.09 (d, J=7.8 Hz, 1H, H-2); ¹³C NMR (126 MHZ, CD₃OD) *41.1, 45.7, 56.6, 62.2, 96.9, 102.4, 113.2, 117.8, 132.3, 133.8, 133.9, 135.3, 148.9, 149.4, 158.1; and EIMS m/z 257 (1) [M⁺-I], 242 (60), 227 (83), 199 (40), 184 (37), 142 (100), 127 (50), 28 (46); HRFABMS m/z [M-7]⁺257.1274 (calcd for C₁₅H₁₇N₂O₂, 257.1290).

Part B. X-Ray Crystal Structure Determination. 1,4-dimethyl-8,9-dimethoxy-4H-benzo-[de][1,6]naphthyridin-1-ium iodide (5). A thin plate (˜0.32×0.16×0.02 mm), grown from a methanol:ethyl acetate solution, was mounted on the tip of a glass fiber. Cell parameter measurements and data collection were performed at 296±1° K on a Bruker SMART 6000 diffractometer. Final cell constants were calculated from a set of 884 strong reflections from the actual data collection. Frames of data were collected in the 2 range of 2.39 to 58.74° (−7≦h≦7, −13≦k≦11, 31 20≦1≦20) using 0.40° steps in ω such that a complete coverage of a sphere of reflections was performed. After data collection, an empirical absorption correction was applied with the program SADABS (Blessing, R., Acta Crysta., 1995, A51, 33-8). Subsequent statistical analysis of the complete reflection set using the XPREP (automatic space group determination program in the SHELXTL) program indicated the space group was P_{\overline{1}}.

Crystal data: C₃₀H₃₄I₂N₄O₄, a=7.1462(14) Å, b=11.855(2) Å, c=18.871(4) Å, V=1532.4(5) Å³, α=90.62(3)°, β=100.82(2)°, μ=102.25(3)°, 8=(Cu Kα)−1.54178 Å, ρ_{c}=1.665 g cm⁻³ for Z=2 and F.W.=768.41, F(000)=760. A total of 8617 reflections were collected, of which 3896 were unique (Rᵢₙₜ=0.2014) and considered observed (Iₒ>2σ(Iₒ)). These were used in the subsequent structure solution and refinement with SHELXTL-V5.1.(SHELXTL-Version 5.1 (1997), an integrated suite of programs for the determination of crystal structures from diffraction daa, available from Bruker AXS, Inc., Madison, Wis.) All non-hydrogen atoms for 5 were located using the default settings of that program. Hydrogen atoms were placed in calculated positions, assigned thermal parameters equal to either 1.2 or 1.5 (depending upon chemical type) of the Uiso value of the atom to which they were attached, and then both coordinates and thermal values were forced to ride that atom during final cycles of refinement. All non-hydrogen atoms were refined anisotropically in a full-matrix least-squares refinement process. The final standard residual R₁ value converged to 0.0674 (for observed data) and 0.0802 (for all data). The corresponding Sheldrick R values were wR₂ of 0.1671 and 0. 1752, respectively, and the GOF=0.967 for all data. The difference Fourier map showed residual electron density. The largest difference peak and hole were +1.291 and −1.391 e/Å³, respectively. However, all peaks were within 1 Å of iodide atoms and were consequently attributed to those atoms. Final bond distances and angles were all within acceptable limits.

9-Demethylaaptamine hydrobromide (18). Part A. Aaptamine (1) hydrochloride (0.21 g, 0.79 mmol) was dissolved in 48% HBr (3 mL). After stirring for 45 minutes at 115-120° C. (preheated oil-bath), the solvent was removed in vacuo to leave a brown solid. Addition of CH₂Cl₂—CH₃OH (10:1) led to the precipitation of a yellow solid. Collection by filtration gave 9-demethylaaptamine (18) hydrobromide as a yellow, green amorphous powder (0.15 g, 64%). Further purification was achieved by column chromatography (silica gel, CH₂Cl₂—CH₃OH 6:1): mp 260-264° C. (dec); UV (CH₃OH) 8ₘₐₓ (log ε) 209 (4.16), 244 (4.26), 268 (4.07), 276 (4.02), 316 (3.37), 366 (3.52), 410 (3.47) nm; IR (KBr)ₘₐₓ 3149, 3074, 3020, 1656, 1604, 1552, 1323, 1238 cm⁻¹; ¹H NMR (500 MHZ, CD₃OD) *4.01 (s, 3H, OCH₃), 6.16 (d, J=6.5 Hz, 1H, H-3), 6.71 (d, J−7.3 Hz, 1H, H-6), 6.92 (s, 1H, H-7), 7.02 (d, J=7.3 Hz, 1H, H-5), 7.63 (d, J=6.5 Hz, 1H, H-2); ¹³C NMR (126 MHZ, CD₃OD) *57.1, 98.2, 101.6, 114.6, 118.2, 128.2, 129.7, 129,8, 131.7, 142.6, 151.7, 153.5; and EIMS m/z 214 (100) [M⁺], 200 (60), 199 (42), 171 (70), 142 (25), 96 (30), 94 (28), 82 (55), 80 (60); HRMS [APCI⁺] m/z [M-Br]⁺215.07805 (calcd for C₁₂H₁₁N₂O₂, 215.08206).

Part B. X-Ray Crystal Structure Determination. A thin plate (18, ˜0.60×0.16×0.03 mm), grown from a methylene chloride-methanol-water solution, was mounted on the tip of a glass fiber. Cell parameter measurements and data collection were performed at 293±2° K on a Bruker SMART 6000 diffractometer. Final cell constants were calculated from a set of 2197 reflections from the actual data collection. Frames of data were collected in the θ range of 4.83 to 68.89° (−8≦h≦8, −10≦k≦11, −12≦1<13) using 0.40° steps in ω such that a complete coverage of a sphere of reflections was performed. After data collection, an empirical absorption correction was applied with the program SADABS. Subsequent statistical analysis of the complete reflection set using the XPREP program indicated the space group was P_{\overline{1}}. Crystal data: C₁₂H₁₁Br₁N₂O₂.3 H₂O, a=6.7623(9), b=9.3302(10), C=11.1680(10) Å, α=83.172(6), β=89.717(7), μ=81.060(7)°, V=691.07(13) Å³, 8=(Cu Kα)=1.54178 Å, ρ_{c}=1.678 g cm⁻³ for Z=2 and F.W.=349.19, F(000)=356. A total of 5139 reflections were collected, of which 2374 were unique (Rᵢₙₜ=0.1063) and considered observed (Iₒ>2σ(Iₒ)). These were used in the subsequent structure solution and refinement with SHELXTL-V5.1.²¹ All non-hydrogen atoms for 18 were located using the default settings of that program. Hydrogen atoms were placed in calculated positions, assigned thermal parameters equal to either 1.2 or 1.5 (depending upon chemical type) of the Uiso value of the atom to which they were attached, and then both coordinates and thermal values were forced to ride that atom during final cycles of refinement. All non-hydrogen atoms were refined anisotropically in a full-matrix least-squares refinement process. The final standard residual R₁ value converged to 0.0949 (for observed data) and 0.1 120 (for all data). The corresponding Sheldrick R values were wR₂ of 0.2364 and 0.2493, respectively and the GOF=0.951 for all data. The difference Fourier map showed residual electron density; the largest difference peak and hole being +1.476 and −0.831 e/Å³, respectively. However, all peaks were within 1 Å of bromine atoms and were consequently attributed to those atoms. Final bond distances and angles were all within acceptable limits.

4-Methylaaptamine (4). Part A. Under an argon atmosphere, aaptamine (1) hydrochloride (1.00 g, 3.78 mmol) was suspended in anhydrous tetrahydrofuran (20 mL) and NaHMDS (7.94 mL, 7.94 mmol, 1.0 M in THF) was added at room temperature. The mixture was stirred for 15 minutes at the same temperature and then cooled to −78° C. Next, the mixture was allowed to warm to room temperature and was stirred for another hour. The reaction was terminated with a 1.0 M HCl solution (10 mL), and the solvent was removed in vacuo. The residue was separated by column chromatography (silica gel, CH₂Cl₂—CH₃OH, 6:1). An analytical sample was obtained by crystallization from ethyl acetate-methanol with slow evaporation of the solvent (0.81 g, 77%): mp 225-227° C. (dec.); R_{f} 0.78 (CH₂Cl₂—CH₃OH, 10:1); UV (CH₃OH) 8ₘₐₓ (log ε) 218 (3.91), 237 (3.82), 257 (3.70), 268 (3.65), 277 (3.61), 313 (3.00), 359 (3.11), 394 (3.10) nm: IR (KBr)ₘₐₓ 1653, 1601, 1465, 1321, 1095 cm⁻¹; ¹H NMR (400 MHZ, D₆-DMSO) *3.63 (s, 3H, NCH₃), 3.82 (s, 3H, OCH₃), 3.98 (s, 3H, OCH₃), 6.44 (d, J=7.2 Hz, 1H, H-3), 6.92 (d, J=7.4 Hz, 1H, H-6), 7.16 (s, 1H, H-7), 7.49 (d, J=7.4 Hz, 1H, H-5), 8.00 (d, J=7.2 Hz, 1H, H-2), 12.46 (br s, 1H, NH); ¹³C NMR (101 MHZ, D₆-DMSO) *40.7, 56.5, 60.4, 96.7, 101.2, 113.0, 116.6, 131.3, 131.6, 133.1, 135.4, 142.7, 149.9, 156.3; and EIMS m/z 242 (84) [M⁺], 227 (100) [M⁺-Me], 213 (36), 184 (42), 183 (43), 128 (48), 127 (40), 28 (65); HRFABMS m/z [M-Cl]⁺243.11373 (calcd for C₁₄H₁₅N₂O₂, 243.11335).

Part B. X-Ray Crystal Structure Determination.

8,9-Dimethoxy-4-methyl-4H-benzo[de][1,6]naphthyridin-4-ium chloride hemihydrate (4): A thin, pale-yellow, needle shaped crystal (˜0.50×0.10×0.10 mm), grown from a chloroform-methanol-water solution, was mounted on the tip of a glass fiber. Cell parameter measurements and data collection were performed at 293±2° K on a Bruker SMART 6000 diffractometer. Final cell constants were calculated from a set of 2197 reflections from the actual data collection. Frames of data were collected in the θ range of 4.41 to 68.87° (−8≦h≦8, −13≦k≦14, −22≦1≦22) using 0.400 steps in ω such that a complete coverage of a sphere of reflections was performed. After data collection, an empirical absorption correction was applied with the program SADABS. Subsequent statistical analysis of the complete reflection set using the XPREP program indicated the space group was P_{\overline{1}}. Crystal data: C₁₄H₁₅ClN₂O₂.½H₂O, a=6.9470(1), b=12.1014(1), c=18.7444(1) Å, α=87.296(1), β=79.56, μ=78.624(1)°, V=1519.16(3) Å³, 8=(Cu Kα)=1.54178 Å, ρ_{c}=1.258 g cm⁻³ for Z=4 and F.W. =287.74, F(000)=604. A total of 11994 reflections were collected, of which 5270 were unique (Rᵢₙₜ=0.1429) and considered observed (Iₒ>2σ(Iₒ)). These were used in the subsequent structure solution and refinement with SHELXTL-V5.1. All non-hydrogen atoms for 4 were located using the default settings of that program. Hydrogen atoms were placed in calculated positions, assigned thermal parameters equal to either 1.2 or 1.5 (depending upon chemical type) of the Uiso value of the atom to which they were attached, and then both coordinates and thermal values were forced to ride that atom during final cycles of refinement. All non-hydrogen atoms of 4 could only be refined isotropically in a full-matrix least-squares refinement process. The final standard residual RI value converged to 0.2256 (for observed data) and 0.2280 (for all data). This very high R value was attributed to twinning. Subsequent application of the Gemini software (also available in the Bruker suite of programs), resulted in a reasonable resolution of the twinned data and a final R value of 0.1235 for 869 reflections from one of the twins. The difference Fourier map showed significant residual electron density; the largest difference peak and hole being +5.919 and −1.459 e/Å³, respectively. Part of this residual electron density was due to a molecule of water being shared by two molecules of the parent compound in the asymmetric unit. The remaining major peaks were within ˜2.0 Å of the chlorine atoms themselves and were consequently attributed to these halide ions. Final bond distances and angles were all within acceptable limits and consistent with the structure proposed for 4.

4-Methyl-8,9-dihydroxy-4H-benzo[de][1,6]naphthyridine (7) hydrobromide. The preceding 4-methylaaptamine (4, 0.50 g, 1.79 mmol) was dissolved in 48% HBr (5.0 mL), and the brown solution was stirred for 6 hours at 150° C. (preheated oil-bath). The solution was filtered, and the solvent was removed in vacuo to leave a brown solid which was purified by column chromatography (Sephadex LH-20, CH₃OH). Diphenol 7 was obtained as a yellow amorphous powder (0.40 g, 75%): mp 250-253° C. (dec); UV (CH₃OH) 8ₘₐₓ (log ε) 208 (4.10), 242 (4.28), 273 (4.11), 360 (3.52), 419 (3.37) nm; IR (KBr)ₘₐₓ 1660 (s), 1608 (s), 1240 (s) cm⁻¹; ¹H NMR (400 MHZ, D₆-DMSO) *3.53 (s, 3H, NCH₃), 6.26 (d, J=7.2 Hz, 1H, H-3), 6.76 (d, J=7.4 Hz, 1H, H-6), 6.85 (s, 1H, H-7), 7.25 (d, J=7.4 Hz, 1H, H-5), 7.84 (t, J=6.6 Hz, 1H, H-2), 9.88 (br, s, 1H, OH), 11.02 (br s, 1H, OH), 12.14 (d, J=6.0 Hz, 1H, NH); ¹³C NMR (101 MHZ, D₆-DMSO) *40.3, 95.7, 104.1, 112.6, 116.2, 126.7, 128.9, 129.2, 133.3, 142.1, 149.8, 150.2; and EIMS m/z 214 (100) [M⁺-HBr], 199 (65), 185 (13), 171 (58), 142 (13), 82 (23), 80 (25), 28 (63), HRFABMS m/z [M-Br]⁺215.0818 (calcd for C₁₂H₁₁N₂O₂, 215.08206).

4-Methyl-8-methoxy-9-hydroxy-4H-benzo[de][1,6]naphthyridine (8) hydrobromide. A solution of 4-methylaaptamine (4) hydrochloride (0.20 g, 0.71 mmol) in 48% HBr (3 mL) was stirred at 115-120° C. (preheated oil-bath) for 1 hour (none of the starting material could be detected by TLC). The solvent was removed in vacuo to leave a brown oil, which was separated by column chromatography (silica gel, CH₂Cl₂—CH₃OH, 6:1). The product was obtained as a brown solid (0.18 g, 82%), which was washed with CH₂Cl₂—CH₃OH (20:1). Collection of the solid by filtration gave phenol 8 as a green amorphous powder: mp 252-255° C. (dec); UV (CH₃OH) 8ₘₐₓ (log ε) 209 (3.87), 244 (4.02), 268 (3.85), 316 (3.30), 366 (3.28), 410 (3.24) nm; IR (KBr)ₘₐₓ 3396, 1658, 1618, 1242, 1089 cm⁻¹; ¹H NMR (400 MHZ, D₆-DMSO) *3.56 (s, 3H, NCH₃), 3.97 (s, 3H, OCH₃), 6.29 (d, J 6.4 Hz, 1H, H-3), 6.83 (d, J=7.0 Hz, 1H, H-6), 7.13 (s, 1H, H-7), 7.34 (d, J=7.0 Hz, 1H, H-5), 7.87 (br t, 1H, H-2), 10.15 (s, 1H, OH), 12.19 (s, 1H, NH); ¹³C NMR (101 MHZ, CD₃OD) *41.3, 57.1, 96.5, 101.7, 115.0, 118.4, 128.4, 128.9, 131.7, 134.1, 143.1, 151.7, 152.7; and EIMS m/z 228 (90) [M⁺], 210 (51) [M⁺-H₂O], 185 (51), 170 (28), 96 (63), 94 (67), 28 (100); HRMS [APCI⁺] m/z [M-Br]⁺229.0985 (calcd for C₁₃H₁₃N₂O₂, 229.0977).

4-(4-Methoxy-benzyl)-8,9-dimethoxy-4H-benzo[de][1,6]naphthyridine (16) hydrochloride. Part A. Under an argon atmosphere, aaptamine (1) hydrochloride (1.00 g, 3.78 mmol) was suspended in anhydrous tetrahydrofuran (20 mL), and sodium hexamethyldisilazane (7.94 mL, 7.94 mmol, 1.0 M in THF) was added at room temperature. The mixture was stirred for 15 min at the same temperature and was then cooled to −78° C. Next, 4-methoxybenzyl bromide (0.91 g, 4.54 mmol) in THF (5 mL) was added (dropwise), and the solution was stirred for 3 hours at −78° C. Afterwards, the reaction mixture was allowed to warm to room temperature and stirred for another hour. The reaction was stopped with a 1.0 M HCl solution (10 mL), and the solvent was removed in vacuo. The residue was separated by column chromatography (silica gel, CH₂Cl₂/MeOH, 6:1). Crystallization from ethyl acetate-methanol with slow evaporation of the solvent gave the PMB derivative of 16 as a yellow amorphous powder (0.95 g, 65%): mp 235-237° C. (dec); R_{f} 0.81 (CH₂Cl₂—CH₃OH, 10:1); UV (CH₃OH) 8ₘₐₓ (log ε) 205 (3.97), 241 (4.04), 261 (4.13), 269 (4.14), 359 (3.26), 395 (3.26) nm; IR (KBr): 1595, 1250 cm⁻¹; ¹H NMR (500 MHZ, CD₃OD) *3.76 (s, 3H, OCH₃), 3.93 (s, 3H, OCH3), 4.05 (s, 3H, OCH₃), 521 (s, 2H, NCH₂), 6.45 (d, J=7.3 Hz, 1H, H-3), 6.94 (d, J=9.0 Hz, 2H, Cₐᵣ), 6.97 (d, J=7.5 Hz, 1H, H-6), 7.14 (s, 1H, H-7), 7.25 (d, J=8.5 Hz, 2H, Cₐᵣ), 7.45 (d, J=7.5 Hz, 1H, H-5), 7.83 (d, J=7.3 Hz, 1H, H-2); ¹³C NMR (126 MHZ, CD₃OD) *55.8, 57.0, 57.1, 61.3, 98.2, 102.9, 115.2, 115.7, 118.9, 127.0, 129.7, 132.9, 133.9, 135.0, 135.6, 143.2, 151.7, 158.4, 161.4; and EIMS m/z 348 (27) [M-Cl], 167 (60), 121 (100), 28 (50); HRMS [APCI⁺] m/z {M-Cl]⁺349.153 (calcd for C₂₁H₂₁N₂O₃, 349.15522).

Part B. X-Ray Crystal Structure Determination. (4-methoxybenzyl)-8,9-dimethoxy-4-4H-benzo[de][1,6]naphthyridine hydrochloride (16): A thin, pale-yellow, needle shaped crystal (˜0.26×0.06×0.03 mm), grown from a chloroform-methanol-water solution, was mounted on the tip of a glass fiber. Cell parameter measurements and data collection were performed at 123±2° K on a Bruker SMART 6000 diffractometer. Final cell constants were calculated from a set of 1349 reflections from the actual data collection. Frames of data were collected in the θ range of 4.65 to 68.750 (−16≦h≦14, −22≦k≦22, −8≦1≦8) using 0.396° steps in ω such that a comprehensive coverage of the sphere of reflections was performed. After data collection, an empirical absorption correction was applied with the program SADABS. Subsequent statistical analysis of the complete reflection set using the XPREP program indicated the space group was P2₁/c.

Crystal data: C₂₁H₂₁ClN₂O₃, a=13.7536(13), b=19.0271(17), c=7.1665(6), Å, β=98.060(5), V=1856.9(3) Å³, 8=(Cu Kα)=1.54178 Å, ρc=1.377 g cm⁻for Z=4 and F.W.=384.85, F(000)=808. A total of 9803 reflections were collected, of which 2990 were unique (Rᵢₙₜ=0.1209) and considered observed (Iₒ>2σ(Iₒ)). These were used in the subsequent structure solution and refinement with SHELXTL-V5.1. All non-hydrogen atoms for 16 were located using the default settings of that program. Hydrogen atoms were placed in calculated positions, assigned thermal parameters equal to either 1.2 or 1.5 (depending upon chemical type) of the Uiso value of the atom to which they were attached, and then both coordinates and thermal values were forced to ride that atom during final cycles of refinement. All non-hydrogen atoms were refined anisotropically in a full-matrix least-squares refinement process. The final standard residual R₁ value converged to 0.0860 (for observed data) and 0.1454 (for all data). The corresponding Sheldrick R values were wR₂ of 0.2255 and 0.2458, respectively, and the GOF=0.995 for all data. The difference Fourier map showed residual electron density; the largest difference peak and hole being +1.226 and −0.527 e/Å ³, respectively. However, all peaks were within 1 Å of chlorine atoms and were consequently attributed to those atoms. Final bond distances and angles were all within acceptable limits.

1-Methyl-4-(4-methoxybenzyl)-8,9-dimethoxy-4H-benzo[de][1,6]-naphthyridin-1-ium iodide (17). Part A. To a suspension of 4-(4-methoxybenzyl)-aaptamine (16) hydrochloride (1.00 g, 2.60 mmol) in anhydrous dimethylformamide (50 mL), potassium carbonate (1.80 g, 13.00 mmol) and methyl iodide (0.81 mL, 13.00 mmol) were added at room temperature. After stirring for 12 hours the solution was filtered, and the solvent was removed in vacuo. The oily residue was separated by column chromatography (silica gel, CH₂Cl₂—CH₃OH, 6:1) to give quaternary bromide 17(1.15 g, 94%): mp 189-191° C. (dec); R_{f} 0.72 (CH₂Cl;₂—CH₃OH, 10:1); UV (CH₃OH) 8ₘₐₓ (log ε) 221 (4.37), 262 (4.19), 271 (4.18), 307 (3.46), 413 (364) nm; IR (KBr)ₘₐₓ 1647, 1612, 1568, 1526, 1464, 1348, 1308, 1260, 1097, 1055 cm⁻¹; ¹H NMR (400 MHZ, CD₃OD) * (s, 3H, CH₃), 3.86 (s, 3H, CH₃), 4.06 (s, 3H, CH₃), 4.09 (s, 3H, CH₃), 5.23 (2H, NCH₂), 6.42 (d, J=9.5 Hz, 1H, H-3), 6.93 (d, J=11 Hz, 2H, Cₐᵣ), 7.02 (d, J=9.3 Hz, 1H, H-6), 7.23 (d, J=11 Hz, 2H, Cₐᵣ), 7.24 (s, 1H, H-7), 7.51 (d, J=9.3 Hz, 1H, H-5), 7.79 (d, J=9.5 Hz, 1H, H-2); ¹³C NMR (101 MHZ, CD₃OD) *47.0, 55.8, 57.2, 57.3, 63.0, 98.5, 104.1, 115.4, 115.7, 120.2, 126.9, 129.7, 134.1, 135.5, 136.4, 150.4, 150.8, 160.5, 161.4; and EIMS m/z 363 (4) [M-I], 227 (44), 142 (78), 121 (100), 28 (46); HRFABMS m/z [M-I]⁺363.17295 (calcd for C₂₂H₂₃N₂O₃, 363.17087).

Part B. X-Ray Crystal Structure Determination of Iodide 17: a thick, pale-yellow, plate shaped crystal (˜0.40×0.40×0.35 mm), was grown from a methanol-ethyl acetate-water solution, and mounted on the tip of a glass fiber. Cell parameter measurements and data collection were performed at 296±2° K on an Enraf-Nonius CAD4 diffractometer. Accurate cell dimensions were determined by least-squares fitting of 25 carefully centered reflections in the range of 35°θ<40° using Cu Kα radiation.

Crystal Data: C₂₂H₂₃IN₂O₃.½ H₂O, FW=499.33, triclinic, P_{\overline{1}}, a=12.697(3), b=13.015(3), c=15.203(3) Å, α=68.31(3), β=70.58(3), μ=81.35(3)°, V=2200.4(8) Å³, Z=4, ρ_{c}=1.507 Mg/m³, μ(CuKα)=11.653 mm⁻¹, 8=1.54178 Å. All reflections corresponding to a complete hemisphere (−1≦h≦13, −13≦k≦13, −15≦1≦16) were collected over the range of 0<2θ<110° using the ω)/2θ scan technique. Friedel reflections were also collected (whenever possible) immediately after each reflection. Three intensity control reflections were also measured for every 60 minutes of X-ray exposure time and showed a decay of −15.1% over the course of the collection. A total of 6143 reflections were collected. Subsequent statistical analysis of the complete reflection data set using the XPREP program, verified the space group as P_{\overline{1}}. After Lorentz and polarization corrections, merging of equivalent reflections and rejection of systematic absences, 5274 unique reflections (R(int)=0.1027) remained, of which 3951 were considered observed (Iₒ>2σ(Iₒ)) and was used in the subsequent structure determination and refinement. Linear and anisotropic decay corrections were applied to the intensity data as well as an empirical absorption correction (based on a series of psi-scans) (North, A. C., et al, Acta. Cryst., 1968 A2, 351.) Structure determination was readily accomplished with the direct-methods program SHELXS. All non-hydrogen atom coordinates were located in a routine run using default values in that program. The remaining H atom coordinates were calculated at optimum positions. All non-hydrogen atoms were refined anisotropically in a full-matrix least-squares refinement using SHELXL. The H atoms were included, their Uiso thermal parameters fixed at 1.2 the Uiso of the atom to which they were attached and forced to ride that atom. The final standard residual R₁ value for 17 was 0.1081 for observed data and 0.1292 for all data. The asymmetric unit was found to contain two independent parent molecules and one disordered molecule of water. The goodness-of-fit on F² was 1.251. The corresponding Sheldrick R values were wR₂ of 0.2909 and 0.3145, respectively. A final difference Fourier map showed residual electron density; the largest difference peak and hole being 2.362 and −0.851 e/Å³, respectively. However, the largest peaks were within 1 Å of the iodide atoms and were thus attributed to those atoms. Final bond distances and angles were all within expected and acceptable limits.

1-Methyl-8,9-dimethoxy-1H-benzo[de][1,6]naphthyridine trifluoromethane-sulfonate (14). To a solution of the quaternary ammonium salt 17 (1.10 g, 2.48 mmol) in trifluoroacetic acid (10 mL) at room temperature was added trifluoromethanesulfonic acid (1 mL), and the mixture was stirred at 75° C. The color of the mixture became deep red. After 1 hour the trifluoromethane sulfonic acid was removed (in vacuo) to leave a red oil, which was dissolved in dichloromethane (50 mL). The organic layer was washed successively with 10% NaHCO₃ (50 mL) and brine (50 mL) and dried (anhy. MgSO₄). The solvent was removed (in vacuo) to leave a brown oil, which was separated by column chromatography (silica gel, CH₂Cl₂—CH₃OH, 6:1). The product was obtained as a fine yellow powder (0.68 g, 70%). Fine yellow needles were obtained by crystallization from EtOAc—CH₃OH: mp 253-255° C. (dec); UV (CH₃OH) 8ₘₐₓ (log ε) 206 (4.16), 2.59 (4.31), 315 (3.59), 390 (3.76) nm; IR (KBr)ₘₐₓ 1288, 1246, 1161, 1030, 636 cm⁻¹; ¹H NMR (500 MHZ, CD₃OD) *3.84 (s, 3H, CH₃), 4.03 (s, 3H, CH₃), 4.04 (s, 3H, CH₃), 6.26 (d, J=7.8 Hz, 1H, H-3), 6.86 (d, J=7.5 Hz, 1H, H-6), 7.08 (s, 1H, H-7), 7.23 (d, J=7.5 Hz, 1H, H-5), 7.68 (d, J=7.8 Hz, 1H, H-2); ¹³C NMR (126 MHZ, CD₃OD) *46.55, 57.12, 62.93, 99.35, 103.51, 114.71, 119.14, 121.81 (q, J=318.3 Hz, CF₃SO₃H), 130.01, 135.36, 135.83, 136.01, 149.92, 150.62, 160.88; ¹⁹F NMR (470 MHZ, CD₃OD) *44.06 (s, CF₃SO₃H); and EIMS m/z 242 (67) [M⁺], 227 (100) {M⁺-Me], 184 (55), 28 (49); HRMS [APCI⁺] m/z [M-CF₃SO₃]⁺243.1127 (calcd for C₁₄H₁₅N₂O₂, 243.1134).

Isoaaptamine hydrobromide (2). A solution of 1-methyl-aaptamine trifluoromethanesulfonate (14) (0.62 g, 1.58 mmol) in 48% HBr (5 mL) was stirred at 115-120° C. (preheated oil-bath). After 1 hour, the solvent was removed, and the brown oily residue was separated by column chromatography (silica gel, CH₂Cl₂—CH₃OH, 6:1). Isoaaptamine (2) hydrobromide was obtained as a yellow amorphous powder (0.40 g, 81%): mp 225-227° C. (dec); UV (CH₃OH) 8ₘₐₓ (log ε) 209 (4.19), 246 (4.23), 270 (4.13), 321 (3.61), 414 (3.64) nm; IR (KBr)ₘₐₓ 1647, 1595, 1300, 1244, 1195 cm⁻¹; ¹H NMR (50 MHZ, D₆-DMSO) *3.95 (s, 3H, OCH₃), 4.02 (s, 3H, NCH₃), 6.20 (d, J=7.5 Hz, 1H, H-3), 6.77 (d, J=7.3 Hz, 1H, H-6), 7.12 (s, 1H, H-7), 7.22 (d, J=7.3 Hz, 1H, H-5), 7.73 (d, J=7.5 Hz, 1H, H-2); ¹³C NMR (126 MHZ, D₆-DMSO) *46.0, 56.6, 97.3, 101.5, 113.1, 118.0, 127.8, 129.2, 129.3, 132.2, 149.0, 149.2, 153.5; and EIMS m/z 228 (100) {M⁺], 213 (54) [M⁺-Me], 185 (35), 170 (21), 28 (46).

### DETAILED DESCRIPTION OF INVENTION RELATING TO CONVERSION OF AAPTAMINE TO HYSTATIN 2

Described herein is the conversion of the marine sponge constituent aaptamine (1) to the cancer cell growth inhibitor and antibiotic designated hystatin 2 (10a). Further, herein is described the syntheses of new benzyl derivatives of aaptamine, 9a-c and 1H-benzo[de][1,6]-naphthyridinium salts 10a-c, all readily prepared from aaptamine (1). Also described herein are the results of an initial SAR evaluation of new benzyl-derivatives of aaptamine (1). Single benzylation was found to occur at nitrogen N-4 and led to the formation of the 4-benzyl-aaptamine derivatives 9a-c, whereas double benzylation gave the quaternary 1H-benzo[de][1,6]-naphthyridinium salts 10a-c. The anticancer and antimicrobial properties of these aaptamine derivatives are described. The quaternary ammonium salts 10a (hystatin 2) and 10b exhibited significant inhibitory activity against the murine P388 lymphocytic leukemia and a minipanel of human cancer cell lines. Salts 10a and 10b also had broad spectrum antimicrobial activities and were most potent against Mycobacterium tuberculosis, Neisseria gonorrhoeae and Micrococcus luteus.

Naphthyridinium bromide 10a was selected for further development, and results of an initial cell cycle analysis and a cDNA microarray study with THP-1 cells showed effects consistent with inhibition of the S-phase of cell growth.

Materials and Methods

Commercially available reagents were obtained from Sigma-Aldrich Company, and solvents were distilled prior to use. Aaptamine (1) was isolated from Hymeniacidon sp. as previously described. The benzyl-bromides were prepared according to a synthesis of 3,4,5-trimethoxybenzyl bromide described previously (Pinney, K. G., et al., Synthesis and Biological Evaluation of Aryl Azide Derivatives of Combretastatin A-4 as Molecular Probes for Tubulin, Bioorg. Med. Chem. 2000, 8, 2417-2425). Column chromatography was performed either using flash silica gel from EM Science (230-400 mesh ASTM) or gravity silica (70-230 mesh ASTM), aluminium oxide from Aldrich (activated, neutral, Bockmann I, ˜150 mesh, 58 A) and Sephadex LH-20 from Pharmacia Fine Chemical AB (25-100 μm). Thin-layer chromatography was performed using aluminium oxide plastic sheets (E. Merck). All compounds were visible under UV light (254 nm). Melting points were recorded employing an Electrothermal 9100 apparatus and are uncorrected. The ¹H and ¹³C spectra were obtained using Varian VXR-500 or VXR 400 instruments. Mass spectral data were recorded using a Varian MAT 312 instrument (EIMS), and IR spectra were determined with a Mattson Instruments 2020 Galaxy Series FTIR instrument. All X-ray structure determinations were done on a Bruker AXS SMART 6000 diffractometer.

General Procedure for the Synthesis of Naphthyridines 9a-c:

Under an argon atmosphere, natural aaptamine hydrochloride (1, 1.00 g, 3.78 mmol) was suspended in anhydrous tetrahydrofuran (20 mL), and sodium hexamethyldisilazane (7.94 mL, 7.94 mmol, 1.0 M in THF) was added at room temperature. The mixture was stirred for 15 min at the same temperature and then cooled to −78° C. A solution of the required benzyl bromide (4.54 mmol) in anhydrous tetrahydrofuran (5 mL) was added (dropwise by syringe), and the mixture was stirred for 3 hours at −78° C. Afterwards, the mixture was allowed to warm up to room temperature and stirred for another hour. The reaction was terminated with a 1.0 M HCl solution (10 mL), and the solvent was removed in vacuo. The residue was purified by column chromatography (silica gel, CH₂Cl₂—CH₃OH 6:1). Analytically pure samples were obtained by crystallization from ethylacetate-methanol using slow evaporation of the solvent.

### 4-Benzyl-8,9-dimethoxy-4H-benzo[de][1,6]naphthyridine hydrochloride (9a)

Product: 0.80 g (59%); mp 137-142° C. (dec.); R_{f} 0.80 (CH₂Cl₂—CH₃OH 10:1); UV (CH₃OH) λₘₐₓ (log ε) 207 (4.18), 217 (4.17), 239 (4.21), 259 (4.22), 268 (4.18), 278 (4.15), 357 (3.51), 394 (3.52); IR (KBr) vₘₐₓ 1651, 1597, 1321, 1248,1091; ¹H NMR (500 MHz, CD₃OD) δ 3.91 (s, 3H, OCH₃), 4.03 (s, 3H, OCH₃), 5.27 (s, 2H, NCH₂), 6.37 (d, J=7.0 Hz, 1H, H-3), 6.92 (d, J=7.5 Hz, 1H, H-6), 7.08 (d, J=1.5 Hz, 1H, H-7), 7.33-7.29 (m, 3H), 7.39-7.36 (m, 5H, Cₐᵣ), 7.42 (d, J=7.5 Hz, 1H, H-5), 7.83 (d, J=7.5 Hz, 1H, H-2); ¹³C NMR (126 MHz, CD₃OD) 993δ 57.14, 57.28, 61.29, 98.11, 102.91, 114.98, 118.78, 128.13, 129.49, 130.30, 132.70, 134.02, 135.37, 135.39, 135.75, 143.78, 151.54, 158.16; EIMS m/z 318 (47) {M⁺-HCl], 303 (12), 289 (10), 227 (50), 213 (11), 199 (15), 184 (22), 167 (14), 91 (100), 65 (13), 28 (55).

### 8,9-Dimethoxy-4-(4-methoxy-benzyl)-4H-benzo[de][1,6]naphthyridine hydrochloride (9b)

Yield: 0.95 g (65%); mp 235-237° C. (dec.); R_{f} 0.81 (CH₂Cl₂—CH₃OH 10:1); UV (CH₃OH) λₘₐₓ (log ε) 205 (3.97), 241 (4.04), 261 (4.13), 269 (4.14), 359 (3.26), 395 (3.26); IR (KBr) vₘₐₓ 2839, 1649, 1595, 1516, 1325, 1250, 1089; ¹H NMR (500 MHz, CD₃OD) δ 3.76 (s, 3H, OCH₃), 3.93 (s, 3H, OCH₃), 4.05 (s, 3H, OCH₃), 5.21 (s, 2H, NCH₂), 6.45 (d, J=7.3 Hz, 1H, H-3), 6.94 (d, J=9.0 Hz, 2H, Cₐᵣ), 6.97 (d, J=7.5 Hz, 1H, H-6), 7.14 (s, 1H, H-7), 7.25 (d, J=8.5 Hz, 2H, Cₐᵣ), 7.45 (d, J=7.5 Hz, 1H, H-5), 7.83 (d, J=7.3 Hz, 1H, H-2); ¹³C NMR (126 MHz, CD₃OD) δ 55.80, 56.96, 57.10, 61.28, 98.15, 102.92, 115.21, 115.68, 118.89, 127.02, 129.69, 132.90, 133.89, 134.98, 135.63, 143.24, 151.72, 158.41, 161.35; EIMS m/z 348 (27) [M⁺-HCl], 227 (11), 167 (60), 121 (100), 33 (10), 28 (50).

X-Ray Crystal Structure Determination (9b). 8,9-Dimethoxy-4-(4-methoxy-benzyl)-4H-benzo[de][1,6]naphthyridin-4-ium chloride (9b): A thin, pale-yellow, needle shaped crystal (˜0.26×0.06×0.03 mm), grown from a chloroform-methanol-water solution, was mounted on the tip of a glass fiber. Cell parameter measurements and data collection were performed at 123±2° K on a Bruker SMART 6000 diffractometer. Final cell constants were calculated from a set of 1349 reflections from the actual data collection. Frames of data were collected in the θ range of4.65 to 68.75° (−16≦h≦14, −22≦k≦22, −8≦1≦8) using 0.396° step in ω such that a comprehensive coverage of the sphere of reflections was performed. After data collection, an empirical absorption correction was applied with the program SADABS. Subsequent statistical analysis of the complete reflection set using the XPREP program indicated the space group was P2₁/c.

Crystal data: C₂₁H₂₁ClN₂O₃, a=13.7536(13), b=19.0271(17), c=7.1665(6), A, β=98.060(5), V=1856.9(3) A³, λ=(Cu Kα)=1.54178 A, ρ_{c}=1.377 g cm⁻³ for Z=4 and F.W.=384.85, F(000)=808. A total of 9803 reflections were collected, of which 2990 were unique (Rᵢₙₜ=0.1209), and considered observed (Iₒ>2σ(Iₒ)). These were used in the subsequent structure solution and refinement with SHELXTL-V5.1. All non-hydrogen atoms for 9b were located using the default settings of that program. Hydrogen atoms were placed in calculated positions, assigned thermal parameters equal to either 1.2 or 1.5 (depending upon chemical type) of the Uiso value of the atom to which they were attached, then both coordinates and thermal values were forced to ride that atom during final cycles of refinement. All non-hydrogen atoms were refined anisotropically in a full-matrix least-squares refinement process. The final standard residual R₁ value converged to 0.0860 (for observed data) and 0.1454 (for all data). The corresponding Sheldrick R values were wR₂ of 0.2255 and 0.2458, respectively and the GOF=0.995 for all data. The difference Fourier map showed residual electron density; the largest difference peak and hole being +1.226 and −0.527 e/Å³, respectively. However, all peaks were within 1 A of chlorine atoms and were consequently attributed to those atoms. Final bond distances and angles were all within acceptable limits.

### 8,9-Dimethoxy-4-(3,4,5-trimethoxybenzyl)-4H-benzo[de][1,6]naphthyridine hydrochloride (9c)

Product obtained: 1.0 g (59%); mp 198-200° C. (dec.); R_{f} 0.87 (CH₂Cl₂CH₃OH 10:1); UV (CH₃OH) λₘₐₓ (log ε) 208 nm (4.56), 240 (4.46), 260 (4.48), 269 (4.45), 360 (3.67), 395 (3.69); IR (KBr) vₘₐₓ 1651, 1593, 1460, 1429, 1321, 1246, 1126, 1091, 999; ¹H NMR (500 MHz, CD₃OD) δ 3.72 (s, 3H, OCH₃), 3.78 (s, 6H, 2×OCH₃), 3.94 (s, 3H, OCH₃), 4.05 (s, 3H, OCH₃), 5.21 (s, 2H, NCH₂), 6.49 (d, J=7.0 Hz, 1H, H-3), 6.62 (s, 2H, Cₐᵣ), 6.98 (d, J=8.0 Hz, 1H, H-6), 7.15 (s, 1H, H-7), 7.47 (d, J=8.0 Hz, 1H, H-5), 7.88 (d, J=7.0 Hz, 1H, H-2); ¹³C NMR (126 MHz, CD₃OD) δ 56.78, 57.13, 57.43, 61.09, 61.30, 98.15, 103.02, 105.71, 115.09, 118.86, 131.25, 132.81, 134.02, 135.19, 135.65, 139.27, 143.56, 151.79, 155.27, 158.38, EIMS m/z408 (24) [M⁺-HCl], 181 (100), 28 (46).

X-Ray Crystal Structure Determination (9c).

8,9-Dimethoxy-4-(3,4,5-trimethoxybenzyl)-4H-benzo[de][1,6]naphthyridin-1-ium chloride (9c): A thin plate (˜0.03×0.30×0.32 mm), grown from a chloroform/methanol/water solution, was mounted on the tip of a glass fiber. Cell parameter measurements and data collection were performed at 298±1° K with a Bruker SMART 6000 diffractometer system using Cu Kα radiation. A sphere of reciprocal space was covered using the MULTIRUN technique (SMART for Windows NT v 5. 605, Bruker AXS Inc., Madison, Wis., 2000.) Thus, six sets of frames of data were collected with 0.400 steps in ω, and a last set of frames with 0.40 steps in φ, such that 97.5% coverage of all unique reflections to a resolution of 0.84 A was accomplished.

Crystal Data: C₂₃H₂₄ClN₂O₅.2 H₂O (hydrate), FW=483.96, monoclinic, P2₁/n, a=8.3429(5), b=21.4198(13), c=28.6694(17) A, β=92.436(2)°, V=5118.75(5) A³,Z=8, ρ_{c}=1.256 mg/m³, μ(CuKα)=1.688 mm⁻¹, λ=1.54178 A, F(000)=2056.

A total of 20110 reflections were collected, of which 6738 reflections were independent reflections (R(int)=0. 1407). Subsequent statistical analysis of the data set with the XPREP program indicated the spacegroup was P2₁/n. Final cell constants were determined from the set of the 2345 observed (>2σ(I)) reflections which were used in structure solution and refinement. An absorption correction was applied to the data with SADBS. Structure determination and refinement was readily accomplished with the direct-methods program SHELXTL. All non-hydrogen atom coordinates were located in a routine run using default values for that program. The remaining H atom coordinates were calculated at optimum positions. All non-hydrogen atoms were refined anisotropically in a full-matrix least-squares refinement procedure. The H atoms were included, their Uiso thermal parameters fixed at either 1.2 or 1.5 (depending on atom type) the value of the Uiso of the atom to which they were attached and forced to ride that atom. The final standard residual R₁ value for 9c was 0.1369 for observed data and 0.2550 for all data. The goodness-of-fit on F² was 1.002. The corresponding Sheldrick R values were wR₂ of 0.3624 and 0.3959, respectively. In addition to two parent molecules in the asymmetric cell unit, four molecules of water solvate were also present in each unit. A final difference Fourier map showed some residual electron density; the largest difference peak and hole being +1.712 and −0.551 e/Å³, respectively. However, the principal peaks were within bonding distance of the halide ions and were consequently attributable to these atoms. Final bond distances and angles were all within expected and acceptable limits.

General Procedure for the Synthesis of 10a-c

Natural aaptamine hydrochloride (1, 0.5 g, 1.89 mmol) was dissolved in anhydrous dimethylformamide (50 mL). Potassium carbonate (1.31 g, 9.45 mmol) and the appropriate benzyl bromide (9.45 mmol) were added at room temperature. After stirring for 12 hours at the same temperature the solution was filtered, and the solvent was removed in vacuo to leave a brown oil. The oily residue was separated by column chromatography (silica gel, CH₂Cl₂/CH₃OH 6:1) to give the desired products as bright yellow compounds.

### 1,4-Bis-(benzyl)-8,9-dimethoxy-4H-benzo[de][1,6]naphthyridin-1-ium chloride (10a, hystatin 2)

Isolated yield: 0.63 g (68%); yellow cubes from EtOAc—CH₃OH; mp 175° C. (dec.); R_{f} 0.69 (CH₂Cl₂/MeOH 10:1); UV (MeOH) λₘₐₓ (log ε) 207 (4.60), 220 (4.52), 246 (4.53), 262 (4.58), 273 (4.55), 282 (4.55), 416 (4.02); IR (KBr) vₘₐₓ 1645, 1568, 1456, 1367, 1338, 1303, 1207, 1157, 1101, 1060, 738; ¹H NMR (500 MHz, CD₃OD) δ 3.56 (s, 3H, OCH₃), 4.01 (s, 3H, OCH₃), 5.38 (s, 2H, NCH₂), 5.71 (s, 2H, NCH₂), 6.52 (d, J=8.0 Hz, 1H, H-3), 7.10 (d, J=7.8 Hz, 1H, H-6), 7.17 (d, J=7.5 Hz, 2H, Cₐᵣ), 7.23-7.42 (m, 8H, Cₐᵣ), 7.26 (s, 1H, H-7), 7.59 (d, J=7.8 Hz, 1H, H-5), 7.95 (d, J=8.0 Hz, 1H, H-2); ¹³C NMR (126 MHz, CD₃OD) δ 57.20, 57.85, 61.46, 62.35, 99.16, 104.36, 115.99, 120.46, 127.21, 128.07, 128.94, 129.60, 129.97, 130.38, 134.25, 134.83, 135.23, 135.71, 136.42, 137.93, 150.57, 150.95, 160.36; EIMS m/z 408 (2) [M⁺-HBr], 364 (43), 318 (39), 303 (66), 273 (77), 227 (42), 167 (33), 91 (100), 65 (30), 28 (32).

X-Ray Crystal Structure Determination (10a, hystatin 2).

1,4-Dibenzyl-8,9-dimethoxy-4H-benzo[de][1,6]naphthyridin-1-ium chloride (10a): A thin plate (˜0.40×0.28×0.10 mm), grown from a chloroform/methanol/water solution, was mounted on the tip of a glass fiber. Cell parameter measurements and data collection were performed at 298±1° K with a Bruker SMART 6000 diffractometer system using Cu Kα radiation. A sphere of reciprocal space was covered using the MULTIRUN technique (SMART for Windows NT v5.605; BrukerAXS, Inc. Madison, Wis. 2000). Thus, six sets of frames of data were collected with 0.400 steps in ω, and a last set of frames with 0.40° steps in φ, such that 93.2% coverage of all unique reflections to a resolution of 0.84 A was accomplished.

Crystal Data: C₂₇H₂₅ClN₂O₂.1 H₂O (hydrate), FW=462.96, triclinic,P, a=9.35870(10), b=10.8354(2), c=11.7009(2) A, α=80.7380(10), β=81.5100(10), γ80.9910(10)°, V=1147.46(3) A³, Z=2, ρ_{c}=1.340 mg/m³, μ(CuKα)=1.733 mm⁻¹, λ=1.54178 A, F(000)=488.

A total of 9247 reflections were collected, of which 4002 reflections were independent reflections (R(int)=0.0807). Subsequent statistical analysis of the data set with the XPREP²⁹ program indicated the spacegroup was P. Final cell constants were determined from the set of the 2877 observed (>2σ(I)) reflections which were used in structure solution and refinement. An absorption correction was applied to the data with SADBS. Structure determination and refinement was readily accomplished with the direct-methods program SHELXTL. All non-hydrogen atom coordinates were located in a routine run using default values for that program. The remaining H atom coordinates were calculated at optimum positions. All non-hydrogen atoms were refined anisotropically in a full-matrix least-squares refinement procedure. The H atoms were included, their Uiso thermal parameters fixed at either 1.2 or 1.5 (depending on atom type) the value of the Uiso of the atom to which they were attached and forced to ride that atom. The final standard residual R₁ value for 10a was 0.0818 for observed data and 0.0972 for all data. The goodness-of-fit on F² was 0.997. The corresponding Sheldrick R values were wR₂ of 0.2320 and 0.2478, respectively. In addition to the parent molecule, a molecule of water solvate was also present in the unique cell unit. A final difference Fourier map showed some residual electron density; the largest difference peak and hole being +1.503 and −0.363 e/Å³, respectively. However, the principal peaks were within close proximity of the halide ions and were consequently attributable to these atoms. Final bond distances and angles were all within expected and acceptable limits.

### 8,9-Dimethoxy-1,4-bis-(4-methoxybenzyl)-4H-benzo-[de][1,6]naphthyridin-1-ium bromide (10b)

Realized yield: 0.97 g (89%); R_{f} 0.64 (CH₂Cl₂/CH₃OH 10:1); UV (CH₃OH) λₘₐₓ (log ε) 209 (4.28), 255 (4.05), 261 (4.08), 273 (4.09), 280 (4.06), 307 (3.45), 316 (3.34), 414 (3.60); IR (KBr) vₘₐₓ 1645, 1610, 1566, 1514, 1249; ¹H NMR (500 MHz, CD₃OD) δ 3.64 (s, 3H, OCH₃), 3.72 (s, 3H, OCH₃), 3.76 (s, 3H, OCH₃), 4.02 (s, 3H, OCH₃), 5.26 (s, 2H, NCH₂), 5.64 (s, 2H, NCH₂), 6.54 (d, J=7.5 Hz, 1H, H-3), 6.85 (d, J=9.0 Hz, 2H, Cₐᵣ), 6.94 (d, J=9.0 Hz, 2H, Cₐᵣ), 7.06 (d, J=7.0 Hz, 1H, H-6), 7.13 (d, J=9.0 Hz, 2H, Cₐᵣ), 7.24 (s, 1 H, H-7), 7.25 (d, J=10.0 Hz, 2H, Cₐᵣ), 7.55 (d, J=7.0 Hz, 1H, H-5), 7.96 (d, J=7.5 Hz, 1H, H-2); ¹³C NMR (126 MHz, CD₃OD) δ 56.24, 56.33, 57.71, 57.95, 61.37, 62.84, 99.65, 104.73, 115.80, 116.22, 116.37, 120.98, 127.39, 129.60, 130.09, 130.23, 134.79, 135.24, 135.99, 136.90, 150.69, 151.27, 160.83, 161.48, 161.88; EIMS m/z 468 (2) [M⁺-HBr], 348 (21), 121 (100), 95 (19), 94 (20), 78 (23), 28 (35).

X-Ray Crystal Structure Determination (10b).

8,9-Dimethoxy-1,4-bis-(4-methoxy-benzyl)-4H-benzo[de][1,6]naphthyridin-1-ium bromide (10b): A large, thin plate (˜0.30×0.30×0.20 mm), grown from a chloroform:methanol solution, was mounted on the tip of a glass fiber with Vaseline. Cell parameter measurements and data collection were performed at ambient (27° C.) with a Bruker SMART 6000 diffractometer system using Cu Kα radiation. A sphere of reciprocal space was covered using the MULTIRUN technique. Thus, six sets of frames of data were collected with 0.396° steps in ω, and a last set of frames with 0.396° steps in φ, such that 98.1% coverage of all unique reflections to a resolution of 0.84 A was accomplished.

Crystal Data: C₂₉H₂₉BrN₂O₄.H₂O (hydrate), FW=567.47, triclinic,P, a=9.6217(2), b=10.9327(3), c=13.4568(3) A, α=87.6370(10), β=86.0040(10), γ=69.7300(10)°, V=1324.42(5) A³, Z=2, ρ_{c}=1.423 mg/m³, μ(CuKα)=2.460 mm⁻¹, λ=1.54178 Z, F(000)=588.

A total of 9328 reflections were collected, of which 3728 reflections were independent reflections (R(int)=0.1038). Subsequent statistical analysis of the data set with the XPREP program indicated the spacegroup was C2/c. Final cell constants were determined from the set of the 2933 observed (>2σ(I)) reflections which were used in structure solution and refinement. An absorption correction was applied to the data with SADABS. Structure determination and refinement was readily accomplished with the direct-methods program SHELXTL. All non-hydrogen atom coordinates were located in a routine run using default values for that program. The remaining H atom coordinates were calculated at optimum positions. All non-hydrogen atoms were refined anisotropically in a full-matrix least-squares refinement procedure. The H atoms were included, their Uiso thermal parameters fixed at either 1.2 or 1.5 (depending on atom type) the value of the Uiso of the atom to which they were attached and forced to ride that atom. The final standard residual R₁ value for 10b was 0.0668 for observed data and 0.0762 for all data. The goodness-of-fit on F² was 1.020. The corresponding Sheldrick R values were wR₂ of 0.1672 and 0.1713, respectively. In addition to the parent molecule, a molecule of water was also present in the asymmetric unit. A final difference Fourier map showed some residual electron density; the largest difference peak and hole being +0.637 and −0.875 e/Å³, respectively. However, the principal peaks were within close proximity of the halide ions sites and were consequently attributable to these atoms. Final bond distances and angles were all within expected and acceptable limits.

### 8,9-Dimethoxy-1,4-bis-(3,4,5-trimethoxy-benzyl)-4H-benzo[de][1,6]-naphthyridin-1-ium bromide (10c)

Final product 1.0 g (79%); mp 116-118° C.; R_{f} 0.75 (CH₂Cl₂/CH₃OH 10:1); UV (CH₃OH) λₘₐₓ (log ε) (4.32), 261 (3.99), 273 (3.98), 416 (3.52); IR (KBr) vₘₐₓ 1649, 1591, 1568, 1460, 1124; ¹H NMR (500 MHz, CD₃OD) δ 3.69 (s, 3H, OCH₃), 3.70 (s, 3H, OCH₃), 3.71 (s, 3H, OCH₃), 3.73 (s, 3H, OCH₃), 3.77 (s, 6H, OCH₃), 4.05 (s, 3H, OCH₃), 5.29 (s, 2H, NCH₂), 5.65 (s, 2H, NCH₂), 6.51 (s, 2H, Cₐᵣ), 6.61 (s, 2H, Cₐᵣ), 6.60 (d, J=7.5 Hz, 1H, H-3), 7.11 (d, J=7.5 Hz, 1H, H-6), 7.29 (s, 1H, H-7), 7.61 (d, J=7.5 Hz, 1H H-5), 8.02 (d, J=7.5 Hz, 1H, H-2); ¹³C NMR (126 MHz, CD₃OD) δ 57.18, 57.29, 57.78, 58.52, 61.59, 61.67, 62.87, 99.86, 104.89, 105.89, 106.27, 116.44, 120.87, 131.58, 134.02, 134.81, 135.26, 136.12, 137.02, 139.48, 139.82, 150.76, 151.35, 155.47, 155.80, 160.94; EIMS m/z 588 (1) [M⁺-HBr], 408 (11), 181 (100), 95 (22), 94 (23), 28 (32).

X-Ray Crystal Structure Determination (10c).

8,9-Dimethoxy-1,4-bis-(3,4,5-trimethoxy-benzyl)-4H-benzo[de][1,6]naphthyridin-1-ium bromide (10c): A large, thin plate (˜0.8×0.42×0.06 mm), grown from a methanol/water solution, was mounted on the tip of a glass fiber with Vaseline. Cell parameter measurements and data collection were performed at 123° K (−150° C.) with a Bruker SMART 6000 diffractometer system using Cu Kα radiation. A sphere of reciprocal space was covered using the MULTIRUN technique. Thus, six sets of frames of data were collected with 0.396° steps in ω, and a last set of frames with 0.396° steps in φ, such that 96.4% coverage of all unique reflections to a resolution of 0.84 A was accomplished.

Crystal Data: C₃₃H₃₇BrN₂O₈.2 H₂O (hydrate), FW=705.59, monoclinic, C2/c, a=24.9554(6), b=12.9354(3), c=22.1264(5) A, β=116.0810(10)°, V=6415.3(3) A³, Z=8, ρ_{c}=1.461 mg/m³, μ(CuKα)=2.267 mm⁻¹, λ=1.54178 A, F(000)=2944.

A total of 23034 reflections were collected, of which 5828 reflections were independent reflections (R(int)=0.0610). Subsequent statistical analysis of the data set with the XPREP program indicated the spacegroup was C2/c. Final cell constants were determined from the set of the 4827 observed (>2σ(I)) reflections which were used in structure solution and refinement. An absorption correction was applied to the data with SADBS. Structure determination and refinement was readily accomplished with the direct-methods program SHELXTL. All non-hydrogen atom coordinates were located in a routine run using default values for that program. The remaining H atom coordinates were calculated at optimum positions. All non-hydrogen atoms were refined anisotropically in a full-matrix least-squares refinement procedure. The H atoms were included, their Uiso thermal parameters fixed at either 1.2 or 1.5 (depending on atom type) the value of the Uiso of the atom to which they were attached and forced to ride that atom. The final standard residual R₁ value for 10c was 0.0848 for observed data and 0.1019 for all data. The goodness-of-fit on F² was 0.997. The corresponding Sheldrick R values were wR₂ of 0.2185 and 0.2364, respectively. In addition to the parent molecule, two molecules of water hydrate were found in the asymmetric unit. A final difference Fourier map showed some residual electron density; the largest difference peak and hole being +1.720 and −0.449 e/Å³, respectively. However, the principal peaks were within close proximity of the halide ions and were consequently attributable to these atoms. Final bond distances and angles were all within expected and acceptable limits.

8,9-Dimethoxy-1-(3-hydroxy-4-methoxybenzyl)-4-(3,4,5-trimethoxybenzyl)-4H-benzo[de][1,6]naphthyridin-1-ium bromide (10d)

4-(3,4,5-Trimethoxybenzyl)-aaptamine (9c, 0.40 g, 0.89 mmol) was dissolved in anhydrous dimethylformamide (50 ml). Potassium carbonate (373 mg, 2.70 mmol) and 3-hydroxy-4-methoxybenzyl bromide (0.585 g, 2.70 mmol) were added. The mixture was stirred for 12 hours at room temperature. Filtration and removal of the solvent in vacuo gave a yellow oil that was subjected to column chromatography (silica gel, CH₂Cl₂/CH₃OH 6:1). The product was obtained as a yellow, brown oil. (0.41 g, 73%): R_{f} 0.39 (CH₂Cl₂—CH₃OH 10:1); UV (CH₃OH) λₘₐₓ (log ε) 210 (4.56), 262 (4.29), 273 (4.27), 281 (4.27), 414 (3.79) nm; IR (KBr) v 1647, 1587, 1568, 1510, 1460, 1425, 1244, 1126 cm⁻¹; ¹H NMR (500 MHz, CD₃OD) δ 3.61 (s, 3H, OCH₃), 3.63 (s, 3H, OCH₃), 3.69 (s, 3H, OCH₃), 3.73 (s, 6H, 2×OCH₃), 3.97 (s, 3H, OCH₃), 5.30 (s, 2H, NCH₂), 5.55 (s, 2H, NCH₂), 6.55 (dd, J=1.5 Hz, 8.5 Hz, 1H, Cₐᵣ), 6.62 (d, J=2 Hz, 1H, Cₐᵣ), 6.69 (s, 2H, Cₐᵣ), 6.75 (d, J=8.0 Hz, 1H, H-3), 6.82 (d, J=8.0 Hz, 1H, Cₐᵣ), 7.12 (d, J=7.3 Hz, 1H, H-6), 7.35 (s, 1H, H-7), 7.80 (d, J=7.3 Hz, 1H, H-5), 8.22 (d, J=8.0 Hz, 1H, H-2), 9.07 (s, 1H, OH); ¹³C NMR (126 MHz, CD₃OD) δ 55.59, 55.86, 56.02, 56.63, 58.86, 60.00, 61.37, 98.05, 98.05, 102.98, 104.99, 112.22, 113.75, 114.15, 117.13, 118.53, 129.28, 129.91, 132.46, 133.01, 134.09, 134.82, 137.32, 146.65, 147.19, 148.91, 149.40, 153.24, 158.17; EIMS m/z 544 (4), 394 (11), 213 (18), 181 (100), 137 (18), 95 (16), 94 (17), 28 (38).

Results and Discussion

The 4-benzyl-aaptamines 9a-c were prepared as described previously for the synthesis of 4-methylaaptamine (4). The selective benzylation of aaptamine (1) was achieved using sodium hexamethyldisilazane (NaHMDS) as a base and benzyl bromide, 4-methoxybenzyl bromide or 3,4,5-methoxybenzyl bromide in tetrahydrofuran at −78° C. (See Scheme 5).

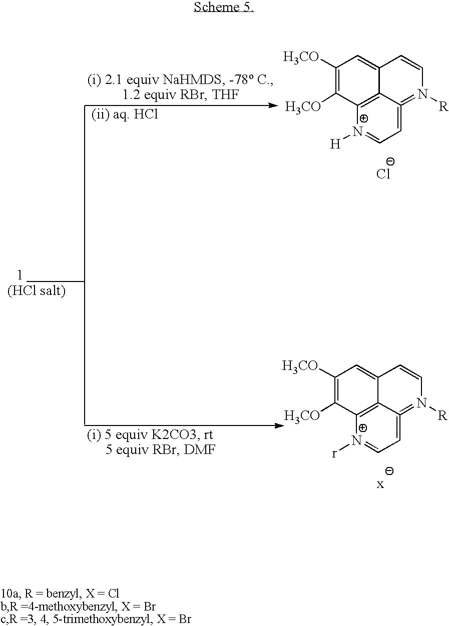

However, an attempt to isolate the free bases of 9a-c using silica gel or alumina column chromatography caused some decomposition. Therefore, when the reaction was complete, dilute hydrochloric acid was added and benzyl amines 9a-c were isolated as stable, greenish yellow hydrochloride salts in yields from 59 to 65%. The X-ray structure analysis of 9b and 9c showed that the benzyl group was bonded to nitrogen N-4 as we observed earlier in the case of 4-methylaaptamine (4).

Fortunately, treatment of aaptamine (1) with potassium carbonate and an excess of benzyl bromide, 4-methoxybenzyl bromide or 3,4,5-methoxybenzyl bromide in dimethylformamide led to formation of the quaternary benzo[de][1,6]-naphthyridinium salts 10a-c in yields from 68 to 89%. These compounds were obtained as bright yellow crystals. Also, we were able to synthesize a naphthyridinium salt with two different benzyl units. For example, treatment of amine 9c with 3-hydroxy-4-methoxy-benzyl bromide led to formation of benzo[de][1,6]-naphthridinium salt 10d (see Scheme 6).

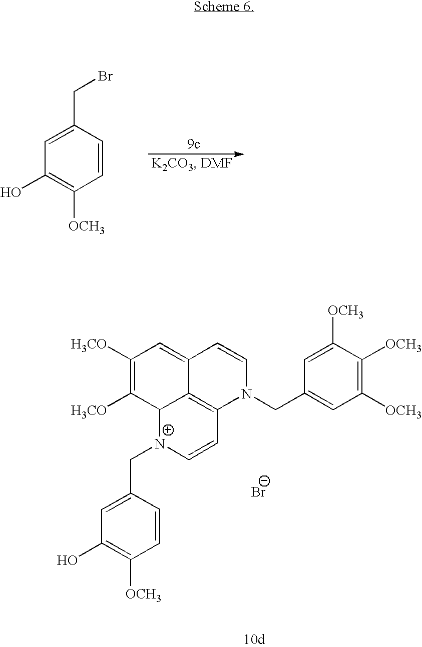

The structures of all these derivatives were established via NMR and high resolution mass spectral data. The structures of the naphthyridinium salts 10a-c were all confirmed by x-ray crystallographic techniques. Surprisingly, naphthyridinium salt 10a crystallized as a chloride salt whereas 10b and 10c crystallized as bromide salts. In every case, we used the hydrochloride salt of aaptamine and the corresponding benzyl bromides. It is noteworthy that 10a crystallized directly from the reaction solution. The chloride salt of 10a was probably less soluble than the corresponding bromide and crystallized preferentially from the reaction solution. Compounds 10b and 10c did not crystallize from the reaction solution and were first purified by column chromatography. These compounds therefore crystallized as bromide salts.

All of the synthetic products were evaluated against a minipanel of human cancer cell lines, the murine P388 lymphocytic leukemia cell line and a selection of microorganisms. These results are summarized in Tables 5 and 6. As previously published, methylation of aaptamine (1) at nitrogen N-4 led to an inactive derivative (4). However, the benzyl derivatives 9a-c exhibited much higher cancer cell line activity than that exhibited by 4-methylaaptamine 4. Within this benzyl series, amine 9b showed good activity, whereas the activity of amine 9c was marginal. The results in the case of the quaternary benzo[de][1,6]-naphthridinium salts 5 and 10a-c were quite similar. The bis-benzyl derivatives 10a and 10b exhibited significant inhibitory activity against the murine P388 lymphocytic leukemia and human cancer cell lines. However, the bis-methyl derivative 5 was less active and salts 10c and 10d were inactive. In general, replacing a methyl-group by a benzyl-group led to an increase in the cancer cell growth inhibitory activity. Additionally, we found that increasing the number of methoxy groups in the series 9a-c and 10a-c led to a decrease in the cancer cell growth inhibitory activity. That was an unexpected result.

Compounds 4, 5, 9a-c, and 10a-d were evaluated as ligands for PKC, based on the initial report of activity for isoaaptamine. Activity was measured at 30 μM compound using inhibition of [20-³H]phorbol 12,13-dibutyrate binding to PKC alpha as described previously (Kazanietz, M. G., et al., Characterization of Ligand and Substrate Specificity for the Calcium-Dependent and Calcium-Independent Protein Kinase C Isoenzymes, Molecular Pharmacol. 1993, 44, 298-307). Assays were carried out with triplicate measurement in either single or triplicate experiments, depending on the compound. Inhibition by 9a and 9b was 6.4% and 9.2%, respectively. That by 10a, 10b, and 10d was 11.8%, 12.6%, and 16.0%, respectively. That by other derivatives was 5% or less. We conclude that these compounds show only very weak activity as ligands for PKC.

We further evaluated the ability of the derivatives to inhibit PKC catalytic activity. PKC alpha was stimulated in the presence of 1 μM phorbol 12-myristate 13-acetate and 100 μg/ml of phospholipid (20% phosphatidylserine: 80% phosphatidylcholine w/w) and its phosphorylation of the peptide PKC selective substrate (cat. number 527151, Calbiochem, La Jolla, Calif.) was determined in the presence of 30 μM compound (Nacro, K., et al., Conformationally Constrained Analogues of Diacylglycero (DAG), 16, How Much Structural Complexity is Necessary for Recognition and High Binding Affinity to Protein Kinase C?, J. Med. Chem., 2000, 43, 921-944.) Marked inhibition was observed for compounds 10a and 10b and lesser inhibition for 10d. Values of percent inhibition were 71.1±4.5%, 84.6±1.1%, and 43.2±10.1%, respectively. Inhibition by 10c was 19.5±7.6% and inhibition by 4, 5, and 9a, b, c was 6% or less (all values are mean±SEM, three experiments). For the most potent compounds, 10a and 10b, we additionally determined complete dose response curves for inhibition. ID₅₀ values were 20.8±2.5 μM and 7.4±1.2 μM, respectively (mean±SEM, 3 experiments). In both cases, the inhibition curves were steeper than predicted for a competitive inhibitor, consistent with a complicated mechanism of action (Hill coefficients for 10a and 10b of 1.5±0.1 and 2.0±0.5, respectively).

A number of the aaptamine derivatives whose synthesis is described here were examined for potential effects on tubulin assembly. These were compounds 2, 4, 5, 9a-c, and 10a-d. No significant activity was observed at the highest concentration evaluated (40 μM), except with compound 2. This agent weakly inhibited the extent of tubulin assembly (20 minutes incubation at 30° C.), with an IC₅₀ value of 31 μM. For comparison, in experiments performed contemporaneously, the potent colchicine site drug combretastatin A-4 yielded an IC₅₀ value of about 2 μM. We therefore conclude that the cytotoxicity of this series of compounds does not result from an interaction with tubulin.

A flow cytometry cell cycle analysis of THP-1 human monocytic leukemia cells, stained with propidium iodide, and pretreated for 24 hours with 0.25 μg/ml 10a, showed an accumulation of cells in the GI phase. A cDNA microarray assay (BD Biosciences Clontech) with THP-1 cells treated for 23 hours with 0.25 μg/ml 10a demonstrated significant down-regulation (5- to 7-fold) of several genes whose products are involved in DNA synthesis. These results suggest that 10a may block the S-phase of the cell cycle.

As set forth below in Table 6, benzyl derivatives 9a-c and bis-benzyl derivatives 10c and 10d had only marginal antibacterial activities. As was the case for cancer cell line inhibition, bis-benzyl derivatives 10a and 10b demonstrated the most promising antibacterial profiles (Table 6). In addition, 10b had antifungal activity (Table 6). Derivatives 9a-c and 10a-d were also evaluated against Mycobacterium tuberculosis. At 6.25 μg/ml, compounds 10a and 10b were active, exhibiting 98% and 97% inhibition, respectively.

In conclusion, convenient syntheses of a selection of benzyl-aaptamines have been developed, many exhibiting significant anticancer and antimicrobial activities, in particular compound 10a.

TABLE 5
Comparative Cancer Cell Line Results (ED₅₀ μg/mL)
for synthetic aaptamines 4, 9a-c, 5 and 10a-d

| Cancer cell line | (4) | (9a) | (9b) | (9c) | (5) | (10a) | (10b) | (10c) | (10d) |
|---|---|---|---|---|---|---|---|---|---|
| leukemia P388 | >10 | 1.64 | 2.19 | 4.64 | 3.89 | 0.234 | 1.30 | >10 | >10 |
| pancreas BXPC-3 | >10 | 4.3 | 4.2 | >10 | 7.1 | 0.061 | 0.11 | >10 | >10 |
| breast MCF-7 | >10 | >10 | 8.0 | >10 | 4.9 | 0.27 | 0.39 | >10 | >10 |
| CNS SF-268 | >10 | >10 | 8.2 | >10 | 0.69 | 0.064 | 0.27 | >10 | >10 |
| lung NCI-H460 | >10 | 4.9 | 5.1 | >10 | >10 | 0.26 | 0.33 | >10 | >10 |
| colon KM 20L2 | >10 | 2.2 | 3.9 | 3.4 | 6.1 | 0.055 | 0.13 | >10 | >10 |
| prostate DRU-145 | >10 | 4.4 | 3.1 | >10 | 3.5 | 0.036 | 0.10 | >10 | >10 |

TABLE 6
Comparative Antimicrobial activities of the synthetic aaptamines 9a-c and 10a-d

| | Range of minimum inhibitory concentration (ug/mL) | | | | | | |
|---|---|---|---|---|---|---|---|
| Microorganism | 9a | 9b | 9c | 10a | 10b | 10c | 10d |
| Cryptococcus neoformans | * | * | * | * | 32-64 | * | * |
| Candida albicans | * | * | * | * | * | * | * |
| Staphylococcus aureus | * | * | * | * | 4 | * | * |
| Streptococcus pneumoniae | 64 | 64 | * | 32 | 16-32 | * | * |
| Enterococcus faecalis | * | * | | 32 | 16-32 | * | * |
| Micrococcus luteus | * | 64 | * | 0.5-2 | <0.5 | 64 | 8-16 |
| Escherichia coli | * | * | * | 8 | * | * | * |
| Enterobacter cloacae | * | * | | * | * | * | * |
| Stenotrophomonas maltophilia | * | * | * | * | * | * | * |
| Neisseria gonorrhoeae | 64 | 64 | 64 | 0.25 | 4 | * | * |

*, no inhibition at 64 μg/mL

1. A derivative of aaptamine selected from the group consisting of: 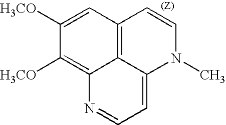
4, 4-N-metbylaaptamine 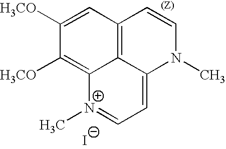
5, 1,4-dimethylaaptamine iodide 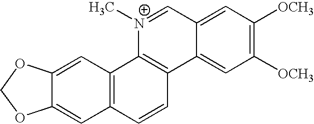
6, nitidine 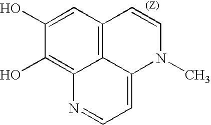
7 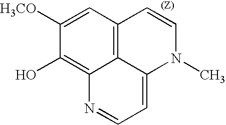
8

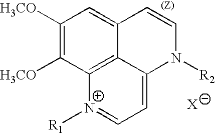

| | R₁ | R₂ | X |
|---|---|---|---|
| 9a | H | benzyl | Cl |
| b | H | 4-methoxylbenzyl | Cl |
| c | H | 3,4,5-trimethoxybenzyl | Cl |
| 10a | benzyl | benzyl | Cl |
| (hystatin | | | |
| 2) | | | |
| b | 4-methoxybenzyl | 4-methoxybenzyl | Br |
| c | 3,4,5-trimethoxybenzyl | 3,4,5-trimethoxybenzyl | Br |
| d | 3-hydroxy-4-methoxybenzyl | 3,4,5-trimethoxybenzyl | Br |

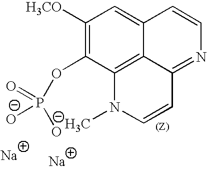

11, hystatin 1 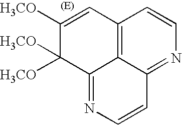
12 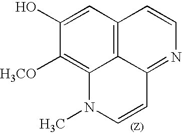
13 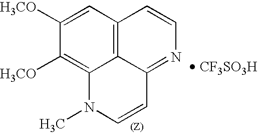
14 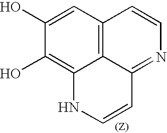
15 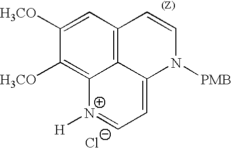
16.

2. A method for converting aaptamine to isoaaptamine, comprising selective O-demethylation of aaptamine at the C-9 position.

3. The method of claim 2, wherein the selective O-demethylation is performed with hydrobromic acid at about 115-120 degrees C.

4. A method for converting aaptamine to 9-demethylaaptamine, comprising the steps of:
a. dissolving aaptamine hydrochloride in hydrogen bromide
b. stirring the solution of step a;
c. removing solvent in vacuo;
d. adding CH₂Cl₁-CH₃OH; and
e. filtering to obtain 9-demethylaaptamine as a solid.

5. A method for converting aaptamine to 4-methylaaptamine, comprising the steps of:
a. suspending aaptamine hydrochloride in anhydrous tetrahydrofuran;
b. adding sodium hexamethyl disilazane at room temperature;
c. stirring, then cooling, then terminating reaction;
d. removing solvent and separating residue via column chromatography.

6. A method for synthesizing hystatin 1 (disodium isoaaptamine 9-0-phosphate) comprising;
a. phosphorylating isoaaptamine with dibenzylphosphate;
b. cleaving the resulting benzyl ester with trimethyl silyl bromide; and
c. reacting the resulting phosphoric acid with sodium methoxide to yield hystatin 1.

7. A method for synthesizing hystatin 2 comprising:
a. dissolving aaptamine hydrochloride in anhydrous dimethyl formamide;
b. adding potassium carbonate and benzyl bromide to form a solution;
c. stirring the solution;
d. filtering the solution;
e. removing solvent in vacuo to obtain an oily residue; and
f. separating the oily residue by column chromatography.

8. A method for treating neoplastic disease comprising administering to a human or animal subject an effective amount of a compound of claim 1.

9. A method for inhibiting the growth of cancer cells comprising administering an effective amount to the cancer cells of a compound, or a physiologically acceptable salt of the compound, selected from the group consisting of:
isoaaptamine;
9-demethoxyloxyaaptamine;
1,4-dimethylaaptamine iodide;
4-Methyl-8,9-dihydroxy-4H-benzo[de][1,6]naphthyridine;
hystatin 2;
1-Methyl-8,9-dimethoxy-1H-benzo[de][1,6]naphthyridine trifluoromethane-sulfonate;
8,9-Dihydroxy-1H-Benzo[de][1,6]naphthyridine hydrobromide;
4-(4-Methoxy-benzyl)-8,9-dimethoxy-4H-benzo[de][1,6]naphthyridine;
1-Methyl-4-(4-methoxybenzyl)-8,9-dimethoxy-4H-benzo[de][1,6]-naphthyridin-1-ium iodide; and
9-demthylaaptamine.

10. A method for treating microbial infections comprising administering to a human or animal subject an effective amount of a compound or a physiologically acceptable salt of the compound, selected from the group consisting of:
Isoaaptamine;
9-demethyloxyaaptamine;
1,4-dimethylaaptamine iodide;
hystatin 2;
8,9-Dihydroxy-1H-Benzo[de][1,6]naphthyridine hydrobromide; and
1-Methyl-4-(4-methoxybenzyl)-8,9-dimethoxy-4H-benzo[de][1,6]-naphthyridin-1-ium iodide

11. A method for inhibiting microbial growth comprising administering a compound or a physiologically acceptable salt of the compound, selected from the group consisting of:
Isoaaptamine;
9-demethyloxyaaptamine;
1,4-dimethylaaptamine iodide;
hystatin 2;
8,9-Dihydroxy-1H-Benzo[de][1,6]naphthyridine hydrobromide; and
1-Methyl-4-(4-methoxybenzyl)-8,9-dimethoxy-4H-benzo[de][1,6]-naphthyridin-1-ium iodide.
